(19) United States
(12) Patent Application Publication
(10) Pub. No.: US 2016/0221941 A1
(43) Pub. Date: Aug. 4, 2016

(54) SELENOCYSTINE DERIVATIVES, ALPHA-METHYLSELENOCYSTEINE, ALPHA-METHYLSELENOCYSTEINE DERIVATIVES, AND METHODS OF MAKING AND USING SAME

(71) Applicant: University of Vermont and State Agricultural College, Burlington, VT (US)

(72) Inventors: Robert J. Hondal, Burlington, VT (US); Erik Ruggles, Huntington, VT (US)

(21) Appl. No.: 15/013,729

(22) Filed: Feb. 2, 2016

Related U.S. Application Data
(60) Provisional application No. 62/110,943, filed on Feb. 2, 2015.

(51) Int. Cl. C07C 391/00; C07K 1/107; C12P 21/06; C07C 227/18

(52) U.S. Cl. CPC C07C 391/00; C07C 227/18; C07K 1/1075; C12P 21/06

(57) ABSTRACT

Provided are selenocysteine derivatives (e.g., deuterium and tritium analogs), α-methylselenocysteine, and α-methylselenocysteine derivatives. These compounds can be incorporated in more complex chemical structures (e.g., polymers, proteins, peptides, and enzymes). Also provided are methods of making and using these compounds. The compounds can be used, for example, as drugs, chemical reagents, and redox switchable surfactants.

### CROSS REFERENCE TO RELATED APPLICATIONS

This application claims priority to U.S. Provisional Application No. 62/110,943, filed on Feb. 2, 2015, the disclosure of which is hereby incorporated by reference.

### STATEMENT REGARDING FEDERALLY SPONSORED RESEARCH

This invention was made with government support under contract no. GM094172 awarded by the National Institutes of Health. The government has certain rights in the invention.

### FIELD OF THE DISCLOSURE

The disclosure generally relates to selenocysteine derivatives. More particularly the disclosure generally relates to α-methylselenocysteine, α-methylselenocysteine derivatives, and methods of making and using α-methylselenocysteine and derivatives thereof.

### BACKGROUND OF THE DISCLOSURE

A number of small molecule antioxidant compounds are now in clinical trials to treat various lung pathologies including COPD (chronic obstructive pulmonary disorders—which include emphysema and chronic bronchitis), cystic fibrosis, idiopathic pulmonary fibrosis, and adult respiratory distress syndrome. Among these are N-acetylcysteine (NAC) and S-carbocysteine (S-CMC) shown in FIG. 1. These also include their lysine salts N-acystelyn and S-CMC-lysine, respectively. Both NAC and S-CMC act as mucolytic agents to help improve lung function by helping to break down the structure of mucin proteins found in mucous. NAC helps to break disulfide bonds between mucin polymers, thereby decreasing the viscosity of mucous. N-acetylcysteine can also act as a glutathione prodrug, thereby acting to increase the concentration of glutathione in the lung, which is exposed to oxidant stress during times when excess mucous is secreted in response to lung infection. S-CMC has been shown to increase the amount of sialomucins, which help to modulate the viscoelastic properties of mucous. NAC and S-CMC only exhibit mucolytic activity and antioxidant capacity when they are in the reduced state, and they cease to be active when oxidized. These molecules contain two distinct disadvantages as antioxidant compounds as shown in FIG. 3. First, they can be irreversibly oxidized to the 2⁻ and 4⁺ oxidation states. Second, these compounds can be destroyed upon oxidation by a reaction called β-elimination.

### SUMMARY OF THE DISCLOSURE

The present disclosure provides compounds having the following structure:

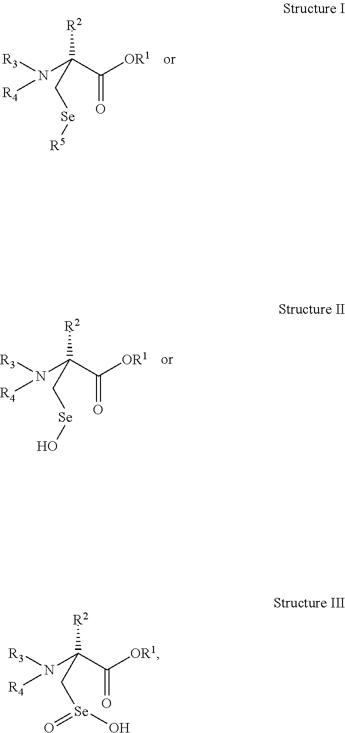

where R¹ is selected from hydrogen atom (H), C₁ to C₁₈ alkyl groups, and protecting group; R² is selected from deuterium atom (D), tritium atom (T), and a methyl group; R³ and R⁴ are each independently selected from hydrogen atom (H), C₁ to C₁₈ aliphatic groups, protecting group, and —C(O)R⁶, where R⁶ is a C₁ to C₁₈ aliphatic group; R⁵ is hydrogen atom (H), protecting group, or CH₂COOR⁷, where R⁷ is H or CH₂R⁸, where R⁸ is an alkyl, aliphatic, or aromatic group.

The present disclosure also provides compounds comprising one or more moiety derived from a compound of the present disclosure (e.g., Structure I, Structure II, or Structure III). For example, the compound is a biological compound selected from the group consisting of polymer, protein, and peptide. In another example, the compound is a lipid conjugated to a compound of the present disclosure or a compound comprising one or more moiety.

The present disclosure also provides compositions comprising one or more compounds of the present disclosure and/or one or more compound comprising one or more moiety derived from a compound of the present disclosure. For example, the composition is an oxidant, acyl transfer group, laundry detergent, surfactant, or food item.

The present disclosure also provides a method of making α-methylselenocysteine, a protected derivative thereof, or a derivative thereof comprising: protecting the amino group and the carboxylate group of α-methylserine to form an α-methylserine having a protected amino group and protected carboxylate group; converting the alcohol group of the α-methylserine to a (alcohol) leaving group to form an α-methylserine having a protected amino group, protected carboxylate group, and leaving group; reacting the α-methylserine having a protected amino group, protected carboxylate group, and leaving group with a selenating reagent comprising a single selenium atom to form a protected α-methylselenocysteine having a protected amino group, protected carboxylate group, and a protected selenol group; and optionally, deprotecting the protected amino group and/or the protected carboxyl group and/or protected selenol group to form α-methylselenocysteine or a protected derivative thereof. The selenating reagent can be formed in situ during the reaction of the α-methylserine having a protected amino group, protected carboxylate group, and (alcohol) leaving group and the selenating reagent. The amino group and/or the carboxylate group of α-methylserine can be protected. Optionally, neither the amino group or the carboxylate group of α-methylserine are protected.

The method can further comprise: forming a diselenide linkage between two of the protected α-methylselenocysteine molecules (e.g., the α-methylserine having a protected amino group, protected carboxylate group, and leaving group above) to form a protected diselenide product; and optionally, deprotecting the protected amino group and/or the protected carboxyl group of the protected diselenide product to form a deprotected diselenide product; reducing the protected diselenide product or deprotected diselenide product with a suitable reagent such that α-methylselenocysteine or carbo-α-methylselenocysteine or a protected derivative thereof is formed. The method can further comprise: forming a —H group or C₁ to C₁₈ ester group by reaction of the protected carboxylate group or deprotected carboxylate group of the α-methylselenocysteine having the protected amino group or deprotected amino group, the protected selenol group or deprotected selenol group, or a combination thereof. The method can further comprise: forming one or two —H groups, one or two C₁ to C₁₈ aliphatic groups, one or two —C(O)R⁶ groups, wherein R⁶ is a C₁ to C₁₈ aliphatic group, or a combination thereof, by reaction of the protected amino group or deprotected amino group of the α-methylselenocysteine having a protected carboxylate group or deprotected carboxylate group, protected selenol group or deprotected selenol group. The method can further comprise: forming a —H group, —CH₂COOR⁷ group, wherein R⁷ is H or CH₂R⁸, wherein R⁸ is an alkyl, aliphatic, or aromatic group, or a combination thereof, by reaction of the protected selenol group or deprotected selenol group of the α-methylselenocysteine having the protected amino group or deprotected amino group, the protected carboxylate group or deprotected carboxylate group. The method can comprise any combination of the foregoing.

The present disclosure also provides a method of making deuterated selenocysteine comprising: transforming α-deuterated serine to α-deutero-β-chloroalanine; and transformation of α-deutero-β-chloroalanine to α-deutero-selenocystine (e.g., by reaction of α-deutero-β-chloroalanine with sodium diselenide). The present disclosure also provides a method of making tritiated selenocysteine comprising: transforming α-tritiated serine to α-tritio-β-chloroalanine; and transforming α-tritio-β-chloroalanine to α-tritio-selenocystine e.g., by reaction of α-deutero-β-chloroalanine with sodium diselenide).

The present disclosure also provides a method of making a composition comprising one or more aqueous soluble peptide fragments of a hydrophobic peptide comprising: forming an aqueous solution or suspension comprising: a compound having the following structure:

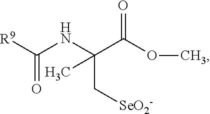

where R₉ is a C₂ to C₁₈ aliphatic group, and a hydrophobic protein (e.g., integral membrane proteins and peripheral membrane proteins), digesting the hydrophobic protein with a protease to form one or more aqueous soluble peptide fragments; and reducing the compound to form a diselenide product and the composition comprising the one or more aqueous soluble peptide fragments of the hydrophobic protein. The method can further comprise analyzing the composition from by mass spectrometry. The method can further comprise: removing the diselenide product from the solution/suspension; forming a seleninic acid analog of the diselenide product; optionally, repeating the removing and forming steps); and optionally, analyzing the composition from by mass spectrometry.

The present disclosure also provides a method of making a disulfide bond comprising contacting a protein and/or peptide that is the same or different that is capable of forming a disulfide bond with a reagent comprising a compound having the following structure:

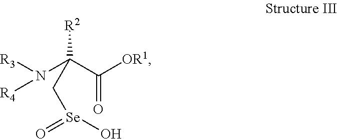

where R¹ is selected from hydrogen atom (H), C₁ to C₁₈ alkyl groups, and protecting group; R² is selected from deuterium atom (D), tritium atom (T), and a methyl group; R³ and R⁴ are each independently selected from hydrogen atom (H), C₁ to C₁₈ aliphatic groups, protecting group, and —C(O)R⁶, where R⁶ is a C₁ to C₁₈ aliphatic group; or a moiety derived from the compound, or a composition comprising the reagent, to form a disulfide bond. The reagent can be coupled to a solid support.

The present disclosure also provides a method of making a thioester bond in a protein or peptide comprising contacting a protein and/or peptide that is the same or different that is capable of forming a thioester bond with a reagent comprising a compound having the following structure:

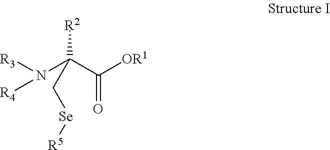

or wherein R¹ is selected from hydrogen atom (H), C₁ to C₁₈ alkyl groups, and protecting group; R² is selected from deuterium atom (D), tritium atom (T), and a methyl group; R³ and R⁴ are each hydrogen atom (H); R⁵ is hydrogen atom (H), or a moiety derived from the compound, or a composition comprising the reagent, to form a thioester bond. The reagent can be coupled to a solid support. The thioester-peptide can be used as a reagent to join (ligate) two peptides. This technology can be referred to as “native chemical ligation” and used to build proteins de novo.

### BRIEF DESCRIPTION OF THE FIGURES

FIG. 1. The structures of N-acetylcysteine (1) and carbocysteine (2). (PRIOR ART)

FIG. 2. An antioxidant compound will react with oxidizing agents, thereby eliminating the harmful effect of the oxidant. A suitable antioxidant will also be able to resists permanent oxidation by being able to be reduced back to the original oxidation state. Both selenium and sulfur react with oxidants such as hydrogen peroxide (H₂O₂) and are converted to higher oxidation states. Selenium is a stronger antioxidant because it reacts with oxygen much faster and it resists permanent oxidation as it can be rapidly reduced back to the original 2⁻ state from both the 0 and 2⁺ oxidation states, whereas reduction of sulfur can only occur from the 0 oxidation state. Critically, reduction back from the 2⁺ state to the parent state is rapid for selenium and slow for sulfur, with the difference in the rate being >10⁶. Each k in the figure represents a rate constant.

FIG. 3. S-CMC has two disadvantages as an antioxidant. First, once it is oxidized to the sulfoxide, it cannot be reduced back to the sulfide under physiological conditions, as indicated by the X. Second, the sulfoxide is destroyed by β-elimination, a reaction by which the hydrogen atom two carbons away (beta to sulfoxide group) is abstracted, resulting in oxidative decomposition of the molecule. (PRIOR ART)

FIG. 4. Selenium analogs of N-acetylcysteine (3) and S-CMS (4) containing the α-methyl selenocysteine building block.

FIG. 5. Description of the lack of beta elimination from (4). The α-methyl group replaces the α-H atom so as to take advantage of selenium's superior antioxidant properties. Now β-elimination is impossible. Unlike a sulfoxide, a selenoxide can be reduced back to the selenide by reducing agents such as vitamin C.

FIG. 6. Further advantages of (4). In addition to be able to be reduced back to the selenide, the selenoxide resists further oxidation to the selenone. It is expected that carbo-α-methylselenocysteine will function as a mucolytic drug, similar to (2). It is expected to be far superior to (2) because it should be a “super” anti-oxidant and anti-inflammatory agent because it is expected to function as a small molecule glutathione peroxidase-mimic. These types of compounds react with oxidants, then are reduced back to the original oxidation state and expel a molecule of water during the redox cycle. This chemical property reduces inflammation.

FIG. 7. Example of incorporation of α-methylselenocysteine into a peptide to provide a Se-peptide based drug. It is expected that such peptide based drugs would have specificity and longer biological half-life.

FIG. 8. Example of use of α-methylselenocysteine or a derivative thereof as an acyl-transfer reagent. It is expected that such a reagent will facilitate the formation of a thioester and allow for two peptides to be ligated together as shown in FIG. 9.

FIG. 9. Example of peptide ligation reaction via a thioester intermediate.

FIG. 10. Example of the use of α-methylselenocysteine derivative as part of a redox-active switchable surfactant. (A) Esterification of α-methylselenocysteine with a fatty acid makes the molecule lipid soluble. After the reaction, it would exist in the diselenide form. (B) Oxidation and hydrolysis of the diselenide form yields a lipid with a polar seleninic acid head group, similar to a phospholipid. In this form, the seleninic acid lipid is a very strong oxidant. (C) After oxidizing a target compound, the lipid would revert back to the hydrophobic diselenide. Regeneration is achieved by oxidation.

FIG. 11. Example of an α-methylselenocysteine glutathione analog. The sulfur containing glutathione is shown at left for comparison.

FIG. 12. The oxidized form of the α-methylselenocysteine glutathione analog is a powerful oxidizing agent that can be used to catalyze the formation of disulfide bonds in peptides and proteins. In this application, it can be attached to a solid support such as, for example, CLEAR™ (cross-linked ethoxylate acrylate resin) as shown in (A). Peptides containing cysteine can be passed over the “Sel-Ox” resin and would elute as oxidized cysteine-containing peptides (B). The “Sel-Ox” resin would become reduced in the process but could be easily be regenerated by the addition of hydrogen peroxide (C).

FIG. 13. ¹H NMR spectrum of sample of 2.

FIG. 14. ¹³C NMR spectrum of sample of 2.

FIG. 15. ¹³C NMR spectrum of sample of 2.

FIG. 16. ¹H NMR spectrum of sample of 3.

FIG. 17. ¹H NMR spectrum of sample of 4.

FIG. 18. ¹³C NMR spectrum of sample of 4.

FIG. 19. ¹³C NMR spectrum of sample of 4.

FIG. 20. ¹³C NMR spectrum of sample of 5.

FIG. 21. ¹³C NMR spectrum of sample of 6.

FIG. 22. ¹³C NMR spectrum of sample of 7.

FIG. 23. ¹H NMR spectrum of sample of 8.

FIG. 24. ¹H NMR spectrum of sample of 9.

FIG. 25. ¹H NMR spectrum of sample of 10.

FIG. 26. ¹H NMR spectrum of sample of 11.

FIG. 27. Example of a synthetic summary of protected α-methyl-selenocysteine.

FIG. 28. α-Methylselenocysteine and derivatives can be methyl esterified and N-acylated with a fatty acid. In the diselenide form, the molecule would be hydrophobic and lipid/organic soluble. Oxidation of the diselenide with, for example, H₂O₂, results in the seleninic acid. In this form, the molecule would have a hydrophilic head group and act as a detergent. The seleninic acid could be easily reduced by, for example, ascorbate (Asc), then air oxidized back to the hydrophobic diselenide. In this way, α-methylselenocysteine and derivatives can be used as a redox-active “switchable surfactants”. For example, α-methylselenocysteine and derivatives can be used as a substitute for cleavable surfactants used in mass spectrometry. α-methylselenocysteine and derivatives has advantages of being recyclable as well as the use of an inexpensive charge silencer (e.g., reductant such as ascorbate). A selenium based surfactant was developed using the reversibility of an alkyl selenide/selenoxide.

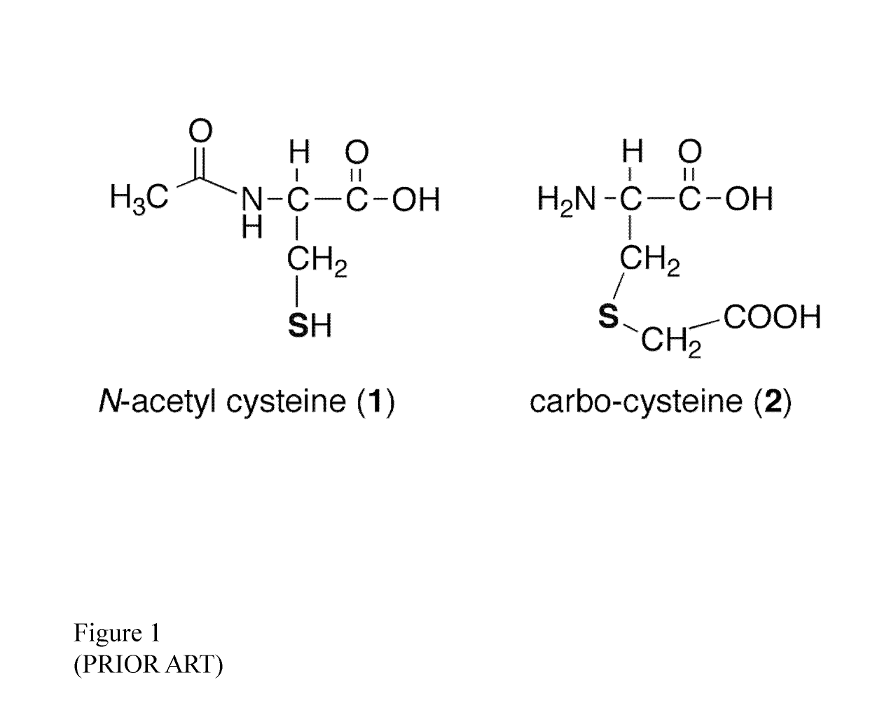

### DETAILED DESCRIPTION OF THE DISCLOSURE

The present disclosure provides selenocysteine (e.g., deuterium/tritium analog), α-methylselenocysteine, derivatives of α-methylselenocysteine, and compounds comprising selenocysteine (e.g., deuterium/tritium analog) α-methylselenocysteine, derivatives of α-methylselenocysteine, and/or moieties derived therefrom. Also, provided are methods of making and methods of using selenocysteine (e.g., deuterium/tritium analog), α-methylselenocysteine, and derivatives of α-methylselenocysteine.

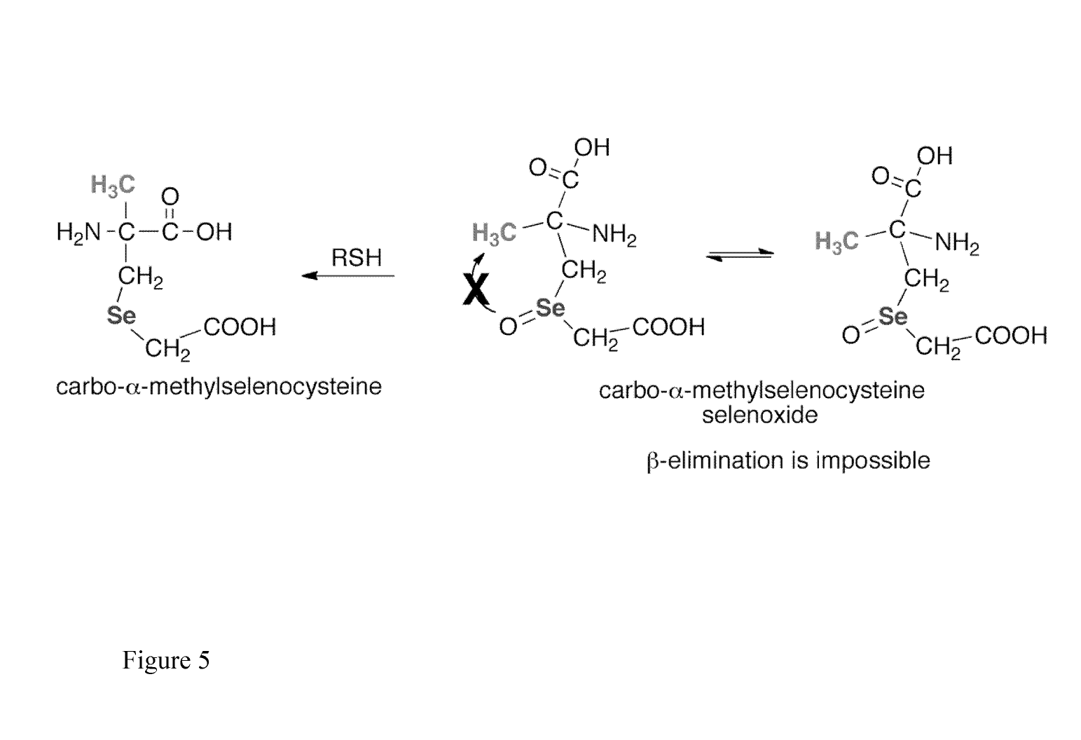

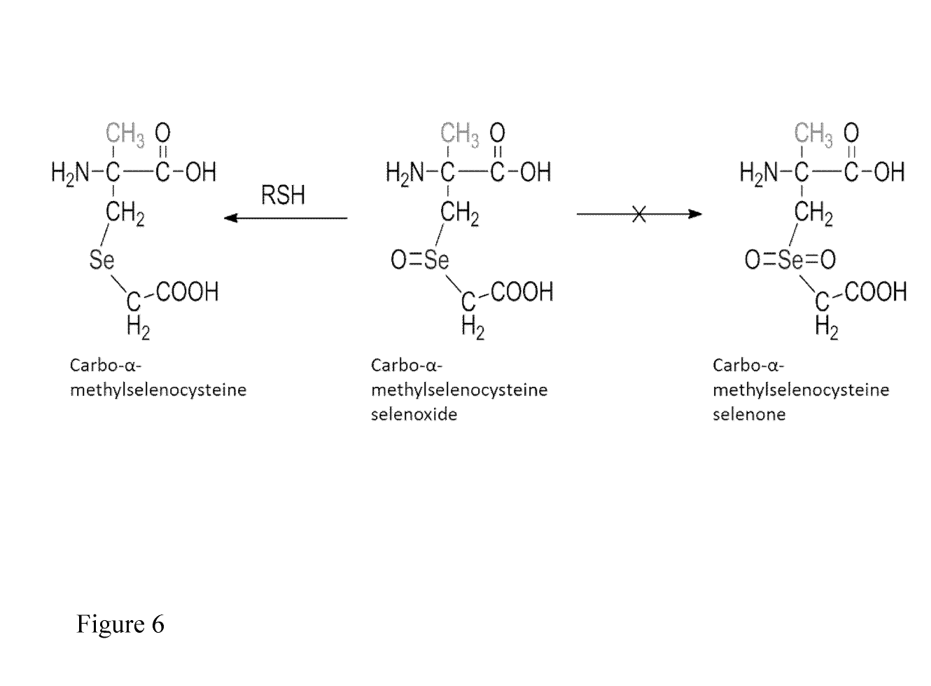

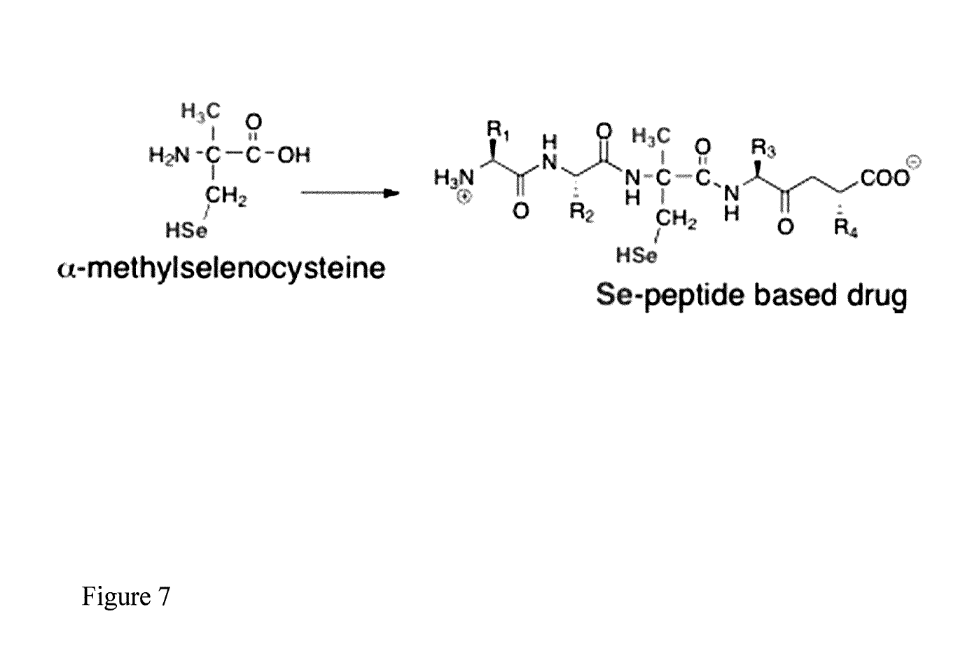

Oxidation of selenols to Se-oxides in the 0 and 2⁺ oxidation states is rapid. Equally rapid is the reduction of the 0 and 2⁺ oxidation states of these Se-oxides back to the parent selenol. Based on the redox property of selenium and its oxides, compounds of the present disclosure constitute a “redox switch” that is advantageous to numerous processes that depend on reversibility. This reversible redox chemistry is useful for applications where selenium can replace sulfur in antioxidant compounds. Both sulfur and selenium containing antioxidant compounds based on cysteine and selenocysteine skeletons are prone to β-elimination, an oxidative cleavage reaction that irreversibly destroys the compound, thereby reducing their antioxidant capacity. The compounds of the present disclosure addresses this problem by making a modification of the selenocysteine skeleton such that the α-hydrogen of the amino acid is replaced with an α-methyl group, making β-elimination impossible (FIG. 5). This change allows selenium to replace sulfur in numerous compounds, with all of the advantages of reversible redox cycling inherent to the selenium atom, but without the possibility of oxidative destruction by β-elimination.

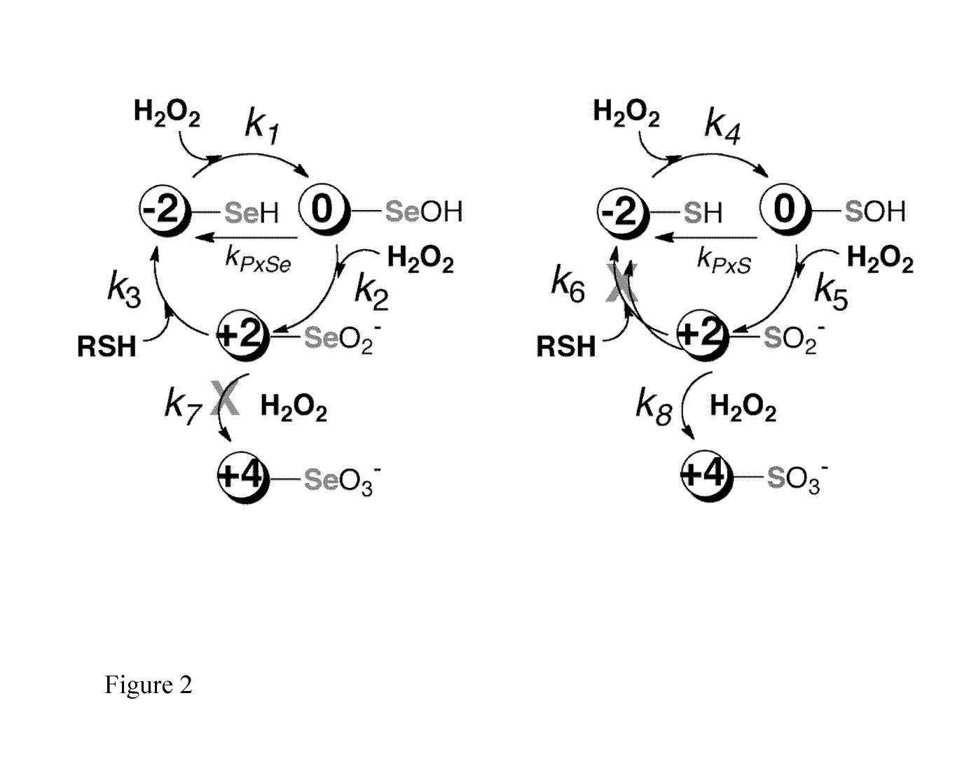

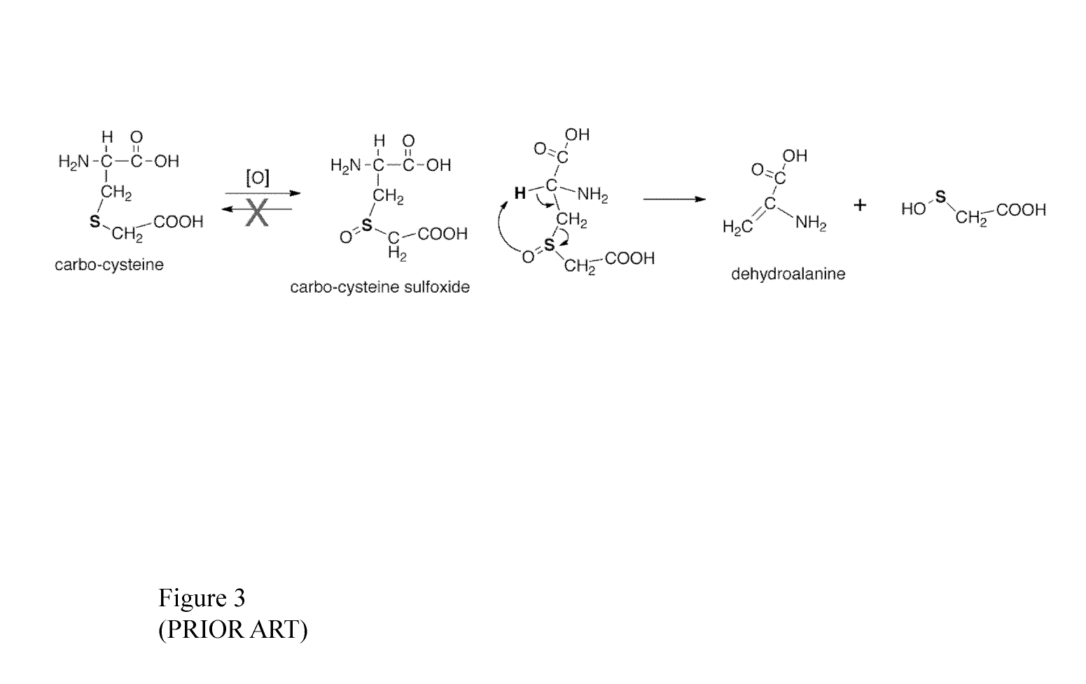

An advantage that selenium has over sulfur is it that it is a superior antioxidant because it reacts with reactive oxygen species, such as hydrogen peroxide, much faster, and importantly, it can also be restored back to the original compound much faster by reaction with reducing agents from both the 0 and 2⁺ oxidation states, whereas sulfur can only be converted back to the original compound from the 0 oxidation state. See, e.g., FIG. 2. Moreover, the reaction from the 2⁺ oxidation state back to the original 2⁻ oxidation state is fast for selenium and is slow for sulfur.

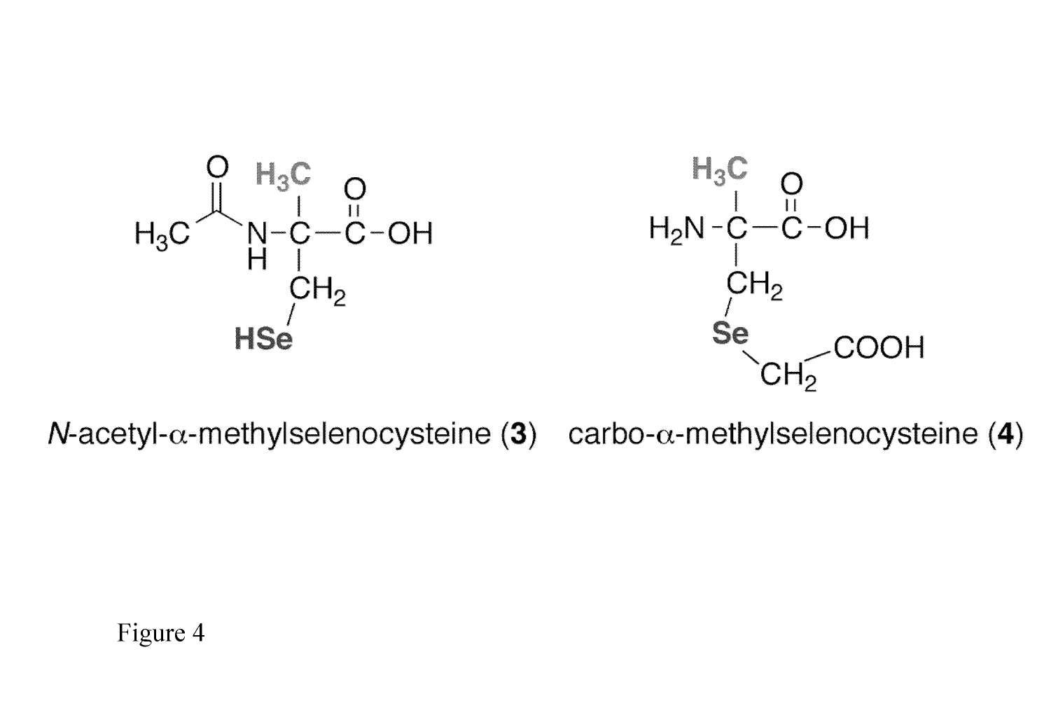

In an aspect, this disclosure provides selenocysteine (e.g., deuterium/tritium analog), α-methylselenocysteine, and derivatives of α-methylselenocysteine. For example, the selenocysteine (e.g., deuterium/tritium analog), α-methylselenocysteine, and derivatives of α-methylselenocysteine are discrete compounds or are moieties of more complex compounds (e.g., moieties in a polymer, protein, peptide or enzyme). Examples of compounds of the present disclosure are shown in FIG. 4. The compounds can be made using one or more of the methods disclosed herein. In an embodiment, the compound is not

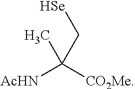

In another embodiment, the compound is not

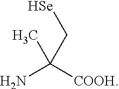

As used herein, the term “alkyl group,” unless otherwise stated, refers to branched or unbranched saturated hydrocarbons derived from alkanes. Examples of such alkyl groups include methyl groups, ethyl groups, propyl groups, butyl groups, isopropyl groups, tert-butyl groups, heptyl groups, nonyl, groups, dodecyl groups, and the like. For example, the alkyl group can be a C₁ to C₁₈ alkyl group including all integer numbers of carbons and ranges of numbers of carbons there between. The alkyl group can be unsubstituted or substituted with various substituents such as: one or more halogens (—F, —Cl, —Br, and —I), the cyano group (—CN), alkoxy groups (—OR), sulfide (—SR), hydroxyl, sulfhydryl (—SH).

As used herein, “aliphatic group” means one carbon atom or a plurality of carbon atoms joined together in straight chains, branched chains, or non-aromatic rings (i.e., alicyclic rings). Aliphatic groups can be saturated, joined by single bonds (alkyl groups), or unsaturated, with one or more double bonds (alkenyl groups) or one or more triple bonds (alkynyl groups). Besides hydrogen, other elements may be bound to the carbon chain (e.g., oxygen, nitrogen, sulfur, and/or chlorine). Examples of aliphatic groups include alkyl groups, alkenyl groups (comprising one or more carbon-carbon double bonds), and alkynyl groups (comprising one or more carbon-carbon triple bonds).

As used herein, the term “aromatic group” refers to unsaturated groups that obey Huckel's Rule for delocalized pi-electrons. Examples of aromatic groups include phenyl groups and substituted phenyl groups substituted with moieties such as, for example: one or more halogens (—F, —Cl, —Br, and/or —I), cyano group (—CN), alkoxy groups (—OR), sulfide groups (—SR), hydroxyl groups, and sulfhydryl group (—SH). Aromatic groups also include heteroaromatic groups containing nitrogen, oxygen and sulfur, that obey Huckel's Rule for delocalized pi-electrons. Examples of heteroaromatic compounds include imidazole groups, thiazole groups, oxazole groups, pyrimidine groups, and pyridine groups, which may be unsubstituted or substituted with moieties such as, for example: one or more halogens (—F, —Cl, —Br, and/or —I), cyano group (—CN), alkoxy groups (—OR), sulfide groups (—SR), hydroxyl groups, and sulfhydryl group (—SH).

In various embodiments, the selenocysteine derivatives, α-methylselenocysteine, and derivatives of α-methylselenocysteine have the following structure:

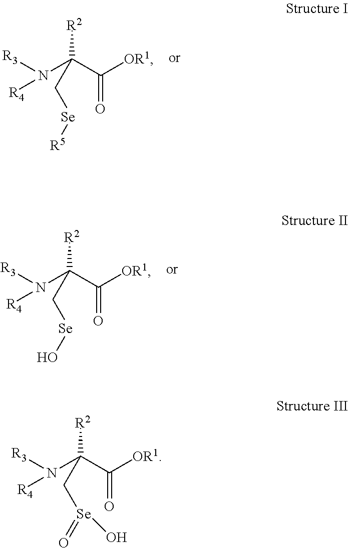

R¹ is selected from hydrogen atom (H), C₁ to C₁₈ alkyl groups, and protecting group. R² is selected from deuterium atom (D), tritium atom (T), and a methyl group. R³ and R⁴ are each independently selected from hydrogen atom (H), C₁ to C₁₈ aliphatic groups, protecting group and —C(O)R⁶, where R⁶ is a C₁ to C₁₈ aliphatic group. For example, the —C(O)R⁶ group is derived from a lipid or fatty acid (e.g., sphingolipids, stearic acid, palmitic acid, glycerols, diacyl glyceric acid, or diacylglycerols, where the acyl group of the diacyl glycerol is C₁ to C₁₈ aliphatic group). R⁵ is hydrogen atom (H), protecting group, CH₂COOR⁷, where R⁷ is H or CH₂R⁸, where R⁸ is an alkyl, aliphatic, or aromatic (e.g., phenyl and substituted phenyl such as 4-methylphenyl, 4-methoxyphenyl) group as defined herein. In an embodiment, Structure I is not α-methylselenocysteine. Structure II is a selenenic acid derivative and Structure III is a seleninic acid derivative. Structures II and III may also exist as their conjugate bases. In another embodiment, R⁷ is not H.

In an embodiment, the compound is selected from the following:

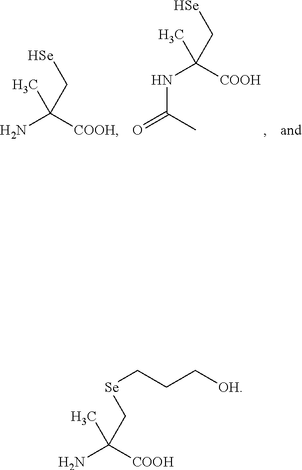

In another embodiment, the α-methylselenocysteine derivative is a seleninic acid having the following structure:

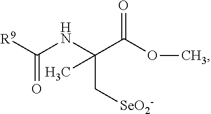

where R⁹ is a C₁ to C₁₈ aliphatic group. For example, R⁹ is a C₂ to C₁₈ aliphatic (e.g., alkyl) group.

The selenocysteine (e.g., deuterium analog), α-methylselenocysteine, and α-methylselenocysteine derivatives may have protecting groups. In an embodiment, the selenocysteine (e.g., deuterium or tritium analog), α-methylselenocysteine, or α-methylselenocysteine derivatives comprises one or more protecting group. When there are more than one protecting group, the protecting groups may be the same or different. Suitable protecting groups are known in the art. Examples of suitable protecting groups include, but are not limited to, acetyl, tosylate, Boc, Fmoc, 4-methoxybenzyl (Mob), 4-methylbenzyl (Meb), diphenylmethyl (Dpm), and benzyl. In an embodiment, the selenocysteine (e.g., deuterium analog), α-methylselenocysteine, or α-methylselenocysteine derivatives comprises one or more protecting groups.

The selenocysteine (e.g., deuterium analog), α-methylselenocysteine, and α-methylselenocysteine derivatives may be part of (e.g., moieties in) more complex compounds (e.g., polymers, proteins, peptides, enzymes, and the like). The more complex compounds may have biological importance and/or activity.

A compound can comprise one or more moieties derived from a compound of the present disclosure. By “moiety derived from a compound” it is meant it is meant that a compound of the present disclosure is covalently bound to a more complex compound, such as, for example, a peptide, polymer, protein, lipid, nucleic acid, or synthetic compound. A compound of the present disclosure can be covalently bound to a more complex compound, such as, for example, a peptide, polymer, protein, lipid, nucleic acid, or synthetic compound, through one or more covalent bonds between the amino group of the α-methylselenocysteine, protected derivative thereof, or derivative thereof, and/or the carboxylate group of the α-methylselenocysteine, protected derivative thereof, or derivative thereof, and/or the selenol group of the α-methylselenocysteine, protected derivative thereof, or derivative thereof, and/or the seleninic acid group of the α-methylselenocysteine, protected derivative thereof, or derivative thereof. Further, the carboxylate group of the α-methylselenocysteine, protected derivative thereof, or derivative thereof, may be coupled with an amine to form an amide or peptide bond. The compound can be a biological compound such as, for example, a polymer, a protein, or a peptide. The compound can be a lipid conjugated to a compound of the present disclosure or a compound comprising one or more moieties derived from a compound of the present disclosure.

In an embodiment, a polymer, lipid, protein, peptide, or enzyme comprises a selenocysteine (e.g., deuterium analog), α-methylselenocysteine, a derivative thereof, or a moiety derived therefrom. For example, a protein or peptide has a selenocysteine (e.g., deuterium analog), α-methylselenocysteine, or α-methylselenocysteine derivative at its C terminus. In an embodiment, a thioredoxin reductase (a thioredoxin reductase derivative) comprises a selenocysteine (e.g., deuterium analog), α-methylselenocysteine, or α-methylselenocysteine derivative thereof. In an embodiment, a glutathione (a glutathione derivative) comprises a selenocysteine (e.g., deuterium analog), α-methylselenocysteine, or α-methylselenocysteine derivative thereof.

As used herein, the terms “peptide”, “polypeptide”, and “protein” are used to compounds comprising a plurality of amino acid resides (i.e., moieties). If a higher order conformation of a polypeptide is stated to be important, then “protein” may indicate the higher order structure while “polypeptide” refers to the amino acid sequence.

As used herein, the terms “detergent” and “surfactant” are used interchangeably to mean an agent that reduces the surface tension of water. For example, a surfactant promotes keeping a hydrophobic polypeptide or generally hydrophobic protein in an aqueous solution.

In various embodiments, the compound(s) of the present disclosure is/are a salt, a partial salt, a hydrate, a polymorph, a stereoisomer or a mixture thereof. For example, the compound(s) can be present as a racemic mixture, a single enantiomer, a single diastereomer, or mixture of diastereomers. In certain embodiments, the compounds are present as mixtures of diastereomers that can be determined by NMR spectroscopy.

In an aspect, the present disclosure provides a composition comprising one or more compounds of the present disclosure and/or one or more compounds comprising one or more moieties derived from a compound of the present disclosure. In an embodiment, the composition comprises 50% by weight to 100% by weight of the compound(s), including all integer values and ranges therebetween.

In an aspect, the present disclosure provides pharmaceutical formulations suitable for administration to a human or non-human animal in need thereof. The formulations comprise one or more compounds of the present disclosure. The pharmaceutical formulations may further comprise one or more pharmaceutically acceptable carrier and/or excipient. Examples of suitable pharmaceutically acceptable carriers and excipients are known in the art. Some examples of pharmaceutically acceptable carriers and excipients can be found in: Remington: The Science and Practice of Pharmacy (2005) 21st Edition, Philadelphia, Pa. Lippincott Williams & Wilkins.

In an aspect, this disclosure provides methods of making selenocysteine (e.g., deuterium analog), α-methylselenocysteine, or α-methylselenocysteine derivatives. Examples of such methods are provided in the examples and related figures.

The deuterated analog of selenocysteine may be prepared by using α-deuterated serine as a starting material, then transforming this to α-deutero-β-chloroalanine, for example, by reaction with PCI₅. The α-deutero-β-chloroalanine compound can then be converted to α-deutero-selenocystine by, for example, addition of disodium diselenide that is prepared in situ by reduction of elemental selenium by sodium borohydride. α-Methylselenocysteine can be prepared by using α-methylserine as the starting material. The amino group of α-methylselenocysteine can be protected, for example, with the Fmoc group and the carboxylate group can be protected, for example, as the methyl ester. Once the amino and carboxyl groups are appropriately protected, the selenol of α-methylselenocysteine can be converted to the O-tosylate. The tosyl group can then be displaced by, for example, 4-methoxybenzylselenolate to yield the fully protected compound. The Mob group can then be removed, for example, by reaction with dithionitropyridine in dilute trifluoroacetic acid to yield the diselenide. The diselenide of α-methylselenocysteine can then be reduced, for example, by sodium borohydride and reacted with iodoacetic acid, resulting in a Se-carboxymethylated molecule. The Fmoc and methyl protecting groups can then be removed by standard procedures to yield carbo-α-methylselenocysteine (4). N-acetyl α-methylselenocysteine can be made similarly except that the amino group would be acetylated, for example, with acetic anhydride and this group is not removed at the end.

The methods may use one or more protecting groups. The protecting groups may be used at one or more steps of the methods. Individual steps of the methods may use multiple protecting groups. When there are more than one protecting group, the protecting groups may be the same or different. Suitable protecting groups are known in the art. Examples of suitable protecting groups include, but are not limited to, acetyl, tosylate, Boc, Fmoc, 4-methoxybenzyl (Mob), 4-methylbenzyl (Meb), diphenylmethyl (Dpm) and benzyl.

In an embodiment, a deuterated analog of selenocysteine is prepared by: a) transforming α-deuterated serine to α-deutero-β-chloroalanine; b) transformation of α-deutero-β-chloroalanine to α-deutero-selenocystine. In an embodiment, a deuterated analog of selenocysteine is prepared by: a) transforming α-tritiated serine to α-tritio-β-chloroalanine; b) transformation of α-tritio-β-chloroalanine to α-tritio-selenocystine.

In an embodiment, α-methylselenocysteine is prepared by: a) protection of the amino group of α-methylselenocysteine; b) protection of the carboxylate group of α-methylselenocysteine; c) optionally, formation of a selenol; d) protection of the selenol; e) formation of a diselenide; f) reduction of the diselenide to form a Se-carboxymethylated compound; g) removal of the amino protecting group and carboxylate protecting group to provide carbo-α-methylselenocysteine. In an embodiment, N-acetyl α-methylselenocysteine is made by the steps in the preceding embodiment, with the exception that the amino group is acetylated and the acetyl group is not removed.

In an embodiment, α-methylselenocysteine is prepared by: a) protecting the amino group of α-methylserine; b) protecting the carboxylate group of a); c)converting the alcohol of b) into a protected selenol; and d) deprotection of the amino protecting group and/or carboxyl protecting group and/or selenol protecting group.

In an embodiment, N-acetyl-α-methylselenocysteine is prepared by: a) protecting the amino group of α-methylserine with an acetyl group; b) protecting the carboxylate group of a); c) converting the alcohol of b) into a protected selenol; and d) deprotection of the carboxyl protecting group and selenol protecting group.

In an embodiment, a method of making α-methylselenocysteine, a protected derivative thereof, or a derivative thereof comprises: a) protecting the amino group, the carboxylate group, and the alcohol group of α-methylserine to form an α-methylserine having a protected amino group and protected carboxylate group; b) converting the alcohol group of the α-methylserine to a leaving group to form an α-methylserine having a protected amino group, protected carboxylate group, and leaving group; c) reacting the α-methylserine having a protected amino group, protected carboxylate group, and protected alcohol group with a selenating reagent (e.g., a selenating reagent comprising a single selenium atom) to form a protected α-methylselenocysteine having a protected amino group, protected carboxylate group, and a protected selenol group; and d) optionally, deprotecting the protected amino group and/or the protected carboxyl group and/or protected selenol group to form α-methylselenocysteine or a protected derivative thereof.

Suitable protecting groups are described herein. A leaving group is a molecular fragment or stable species that can be detached from a molecule in a bond-breaking step. The leaving group, is not particularly limited and should be known to a person having skill in the art. The ability of a leaving group to depart is correlated with the pKa of the conjugate acid, with lower pKa being associated with better leaving group ability. Examples of suitable leaving groups include, but are not limited to sulfonate groups such as nonaflate, triflate, fluorosulfonate, tosylate (e.g., p-tosyl), mesylate and besylate groups.

The selenating reagent reacts with an alcohol group to form a protected silanol group. For example, the selenating reagent is a unsubstituted or substituted benzyldiselenide, such as benzyldiselenide, di-p-methoxybenzylselenide, or di-p-methylbenzylselenide. The selenating reagent can be formed in situ during the reaction of the α-methylserine having a protected amino group, protected carboxylate group, and leaving group and the selenating reagent.

The methods can further comprise formation of derivitized products and/or protected or partially protected products. In an embodiment, the method further comprises: e) forming a diselenide linkage between two of the protected α-methylselenocysteine molecules of c) to form a protected diselenide product; and f) optionally, deprotecting the protected amino group and/or the protected carboxyl group of the protected diselenide product to form a deprotected diselenide product; g) reducing the protected diselenide product or deprotected diselenide product of e) or f) with a suitable reagent such that α-methylselenocysteine or carbo α-methylselenocysteine or a protected derivative thereof is formed. In an embodiment, the method further comprises: h) forming a —H group or C₁ to C₁₈ ester group by reaction of the protected carboxylate group or deprotected carboxylate group of the α-methylselenocysteine having the protected amino group or deprotected amino group, the protected selenol group or deprotected selenol group, or a combination thereof. In another embodiment, the method further comprises: i) forming one or two —H groups, one or two C₁ to C₁₈ aliphatic groups, one or two —C(O)R⁶ groups, wherein R⁶ is a C₁ to C₁₈ aliphatic group, or a combination thereof, by reaction of the protected amino group or deprotected amino group of the α-methylselenocysteine having a protected carboxylate group or deprotected carboxylate group, protected selenol group or deprotected selenol group. In another embodiment, the method further comprising; j) forming a —H group, —CH₂COOR⁷ group, wherein R⁷ is H or CH₂R⁸, wherein R⁸ is an alkyl, aliphatic, or aromatic group, or a combination thereof, by reaction of the protected selenol group or deprotected selenol group of the α-methylselenocysteine having the protected amino group or deprotected amino group, the protected carboxylate group or deprotected carboxylate group. The methods can comprises combinations of these embodiments.

The methods include methods of making polymers, proteins, peptides, or enzymes comprising selenocysteine(s) (e.g., deuterium analog(s)), α-methylselenocysteine(s), or α-methylselenocysteine derivative(s) thereof. In an embodiment, a method of making polymer, protein, or enzyme comprising selenocysteine(s) (e.g., deuterium analog(s)), α-methylselenocysteine(s), or derivative(s) thereof. Methods of making polymers, proteins, peptides, or enzymes known in the art can be used to make the instant polymers, proteins, peptides, or enzymes.

In an aspect, this disclosure provides methods of using selenocysteine (e.g., deuterium analog), α-methylselenocysteine, or α-methylselenocysteine derivatives. For example, these compounds can be used as drugs, reagents in chemical reactions (e.g., oxidants), cleavable surfactants, and redox-active surfactants.

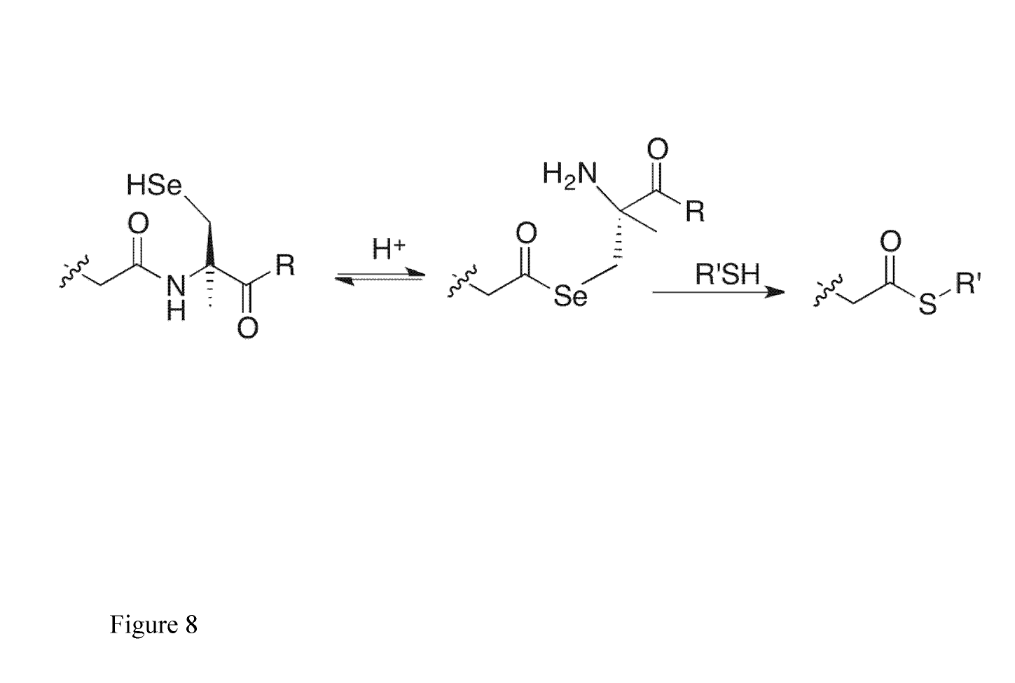

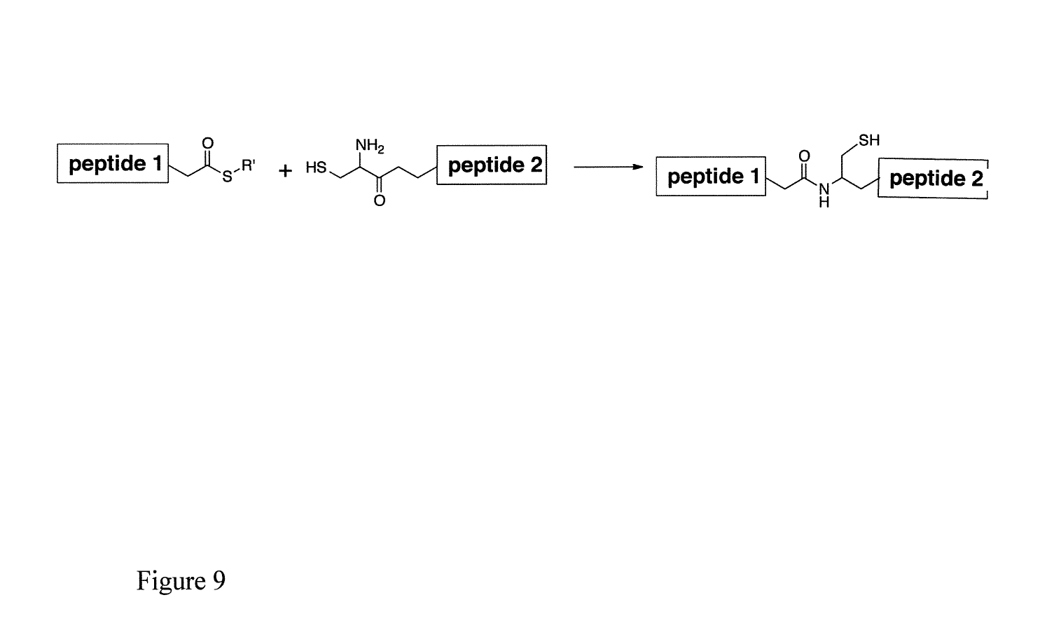

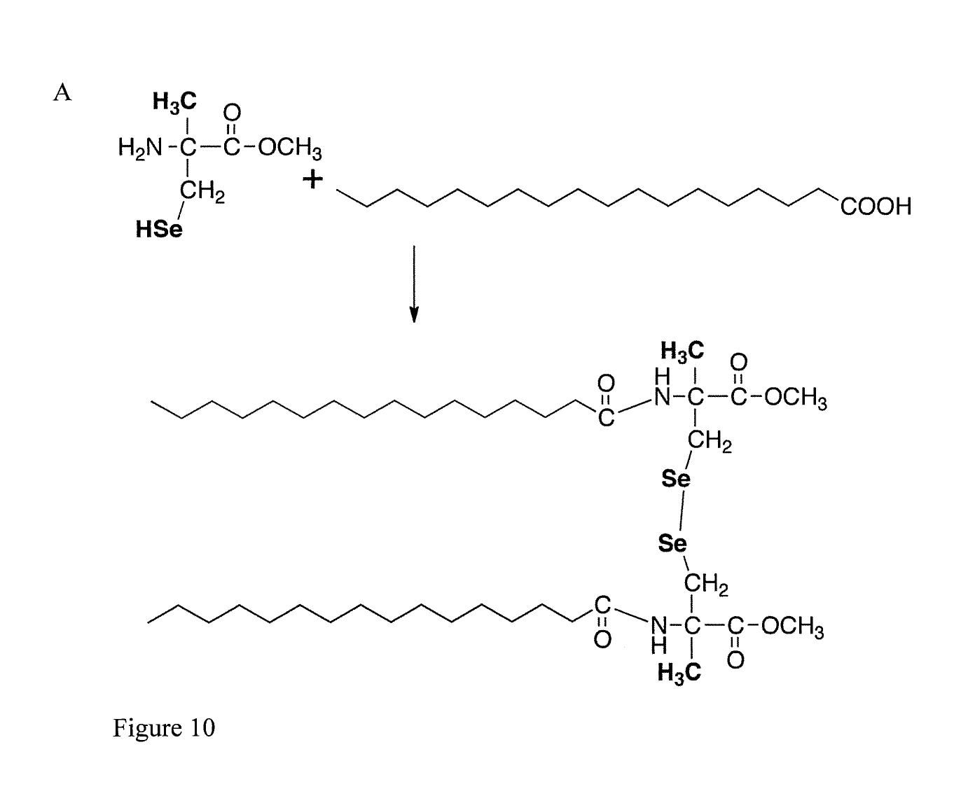

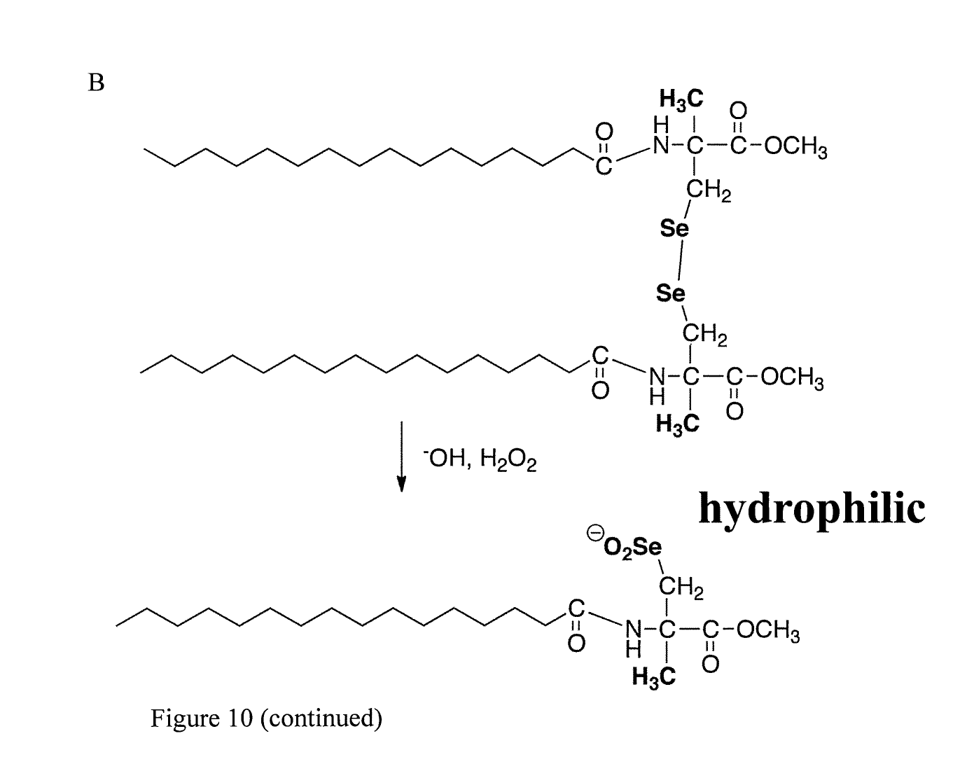

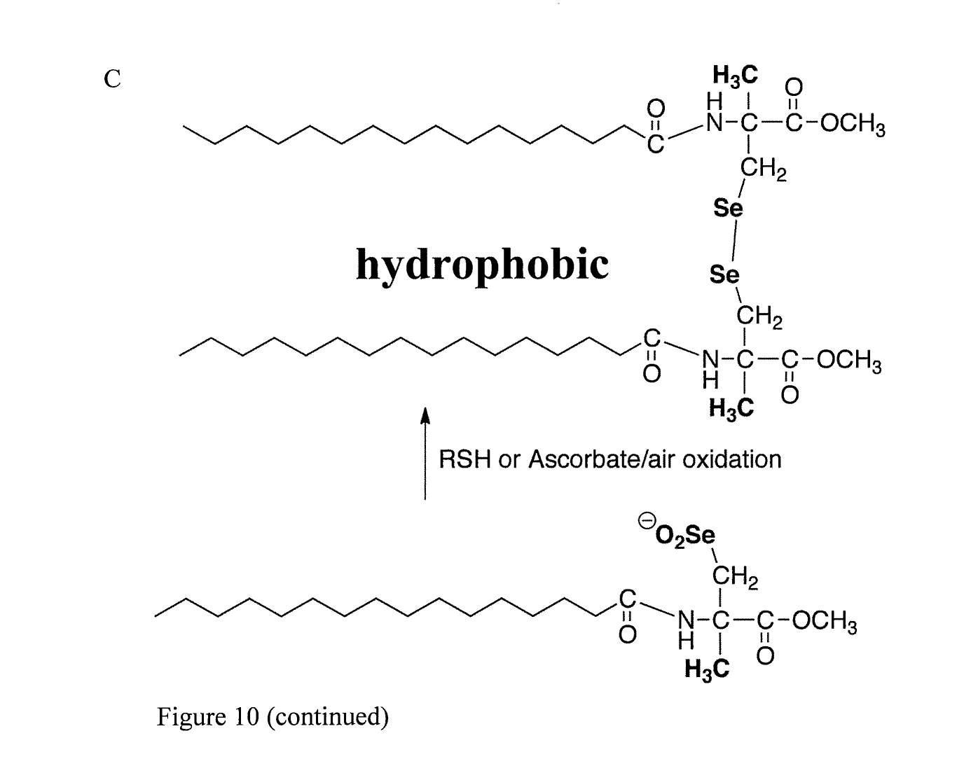

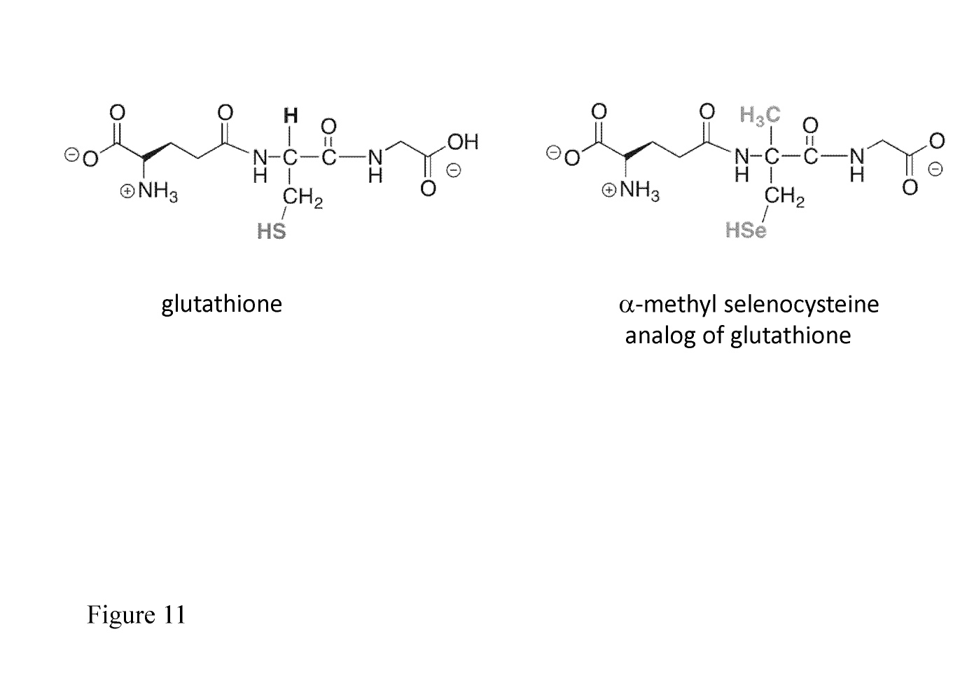

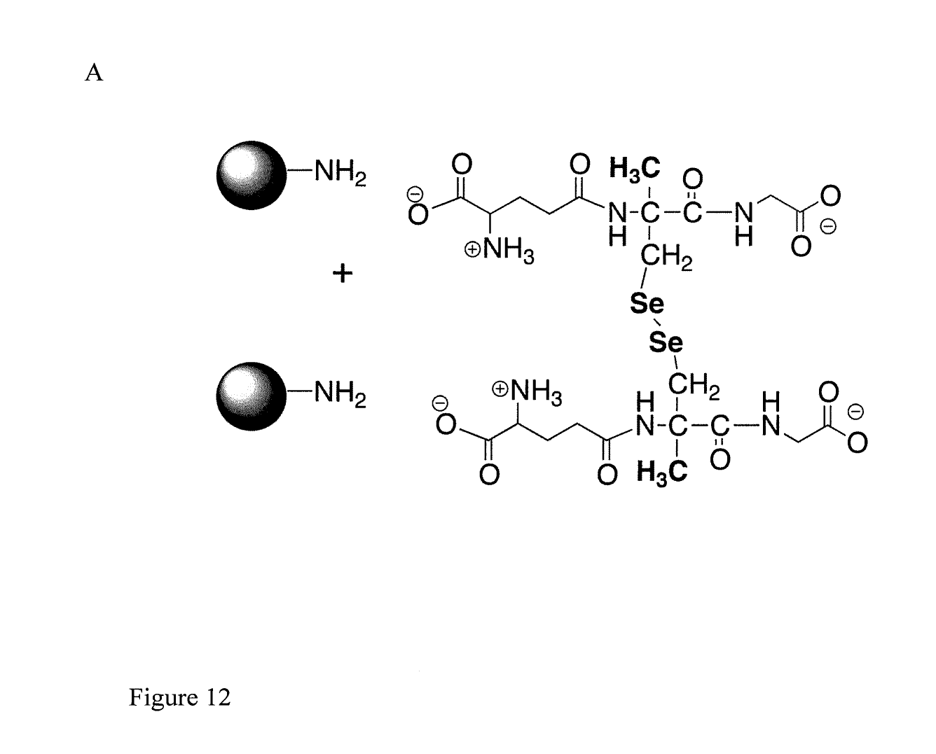

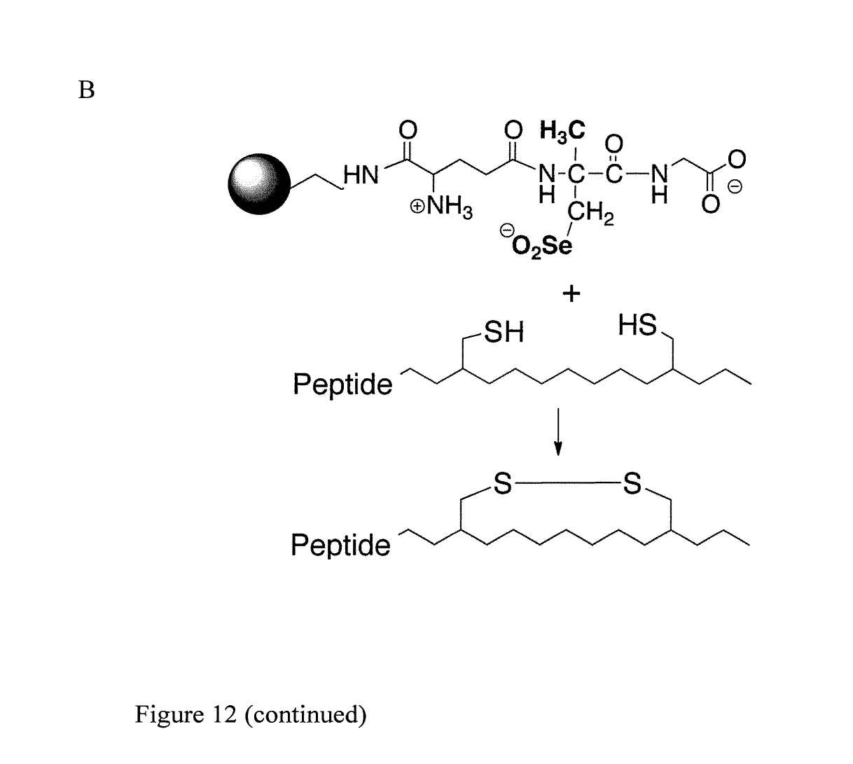

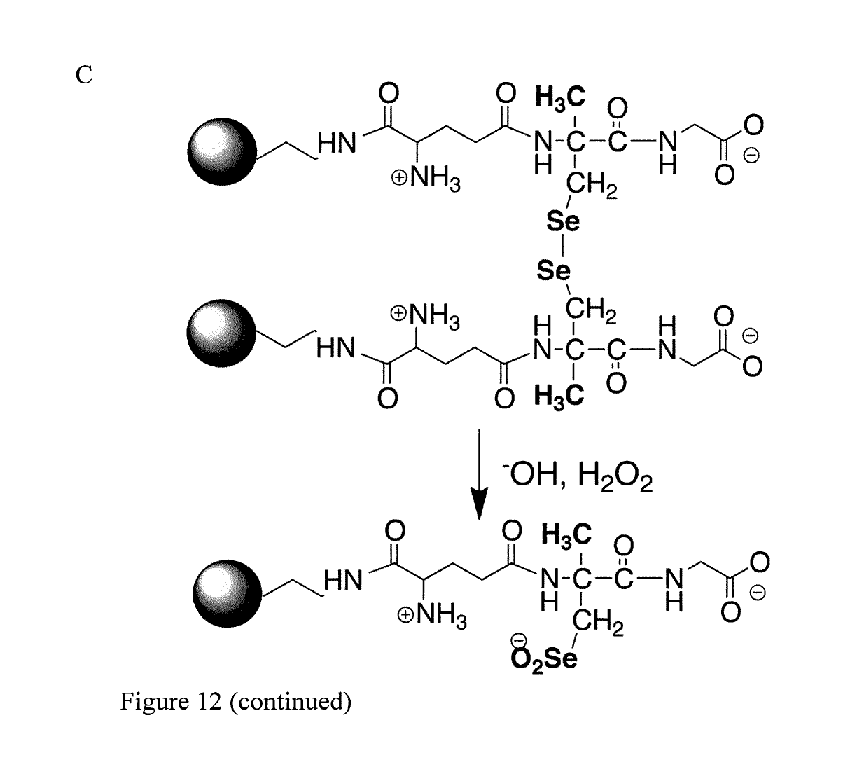

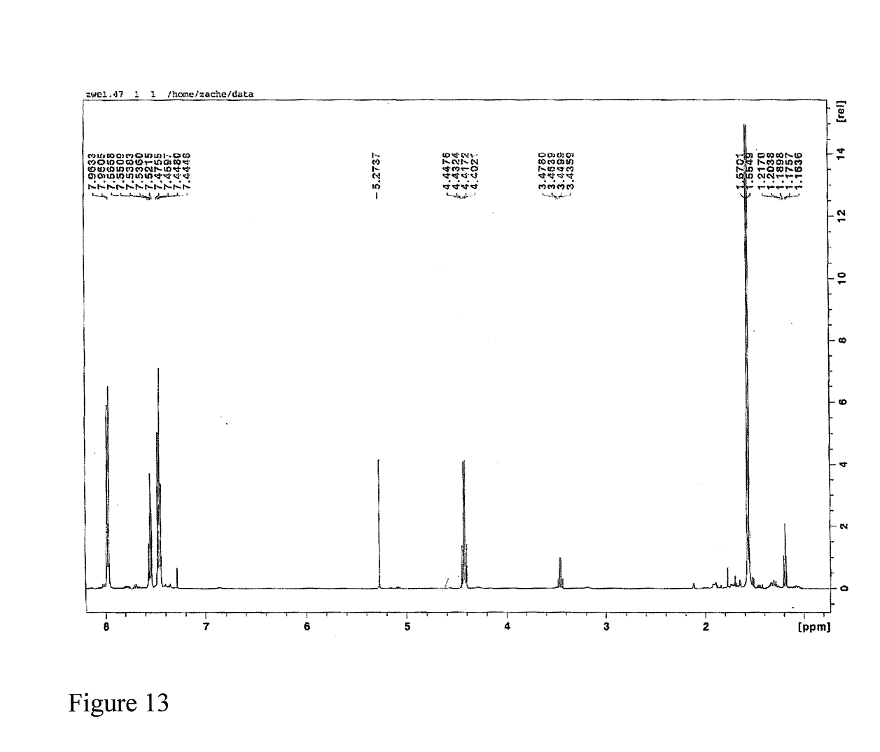

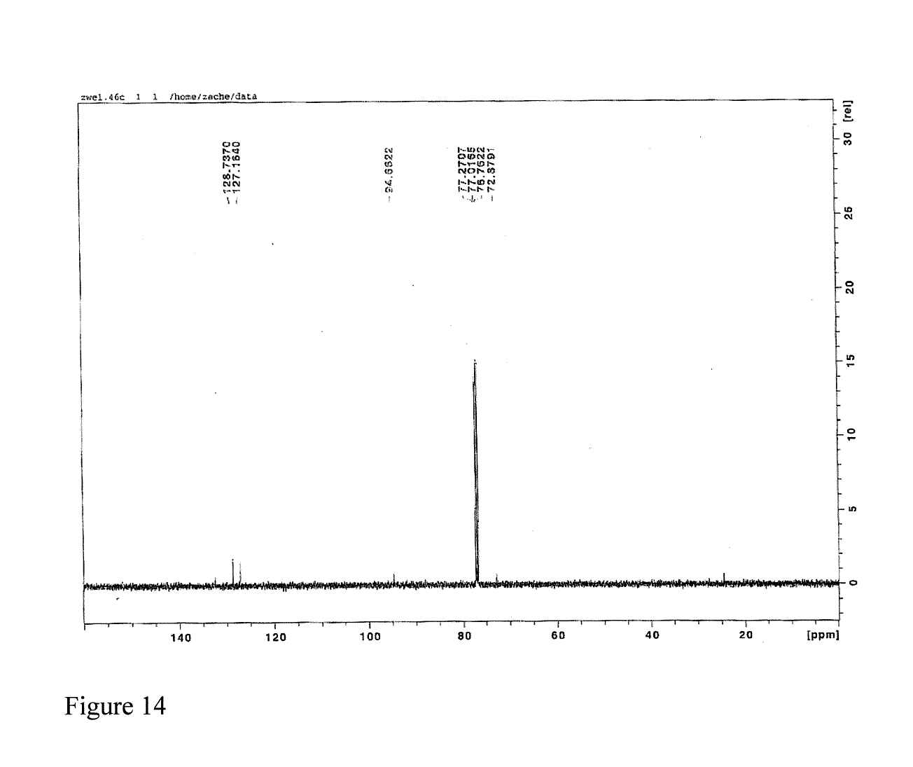

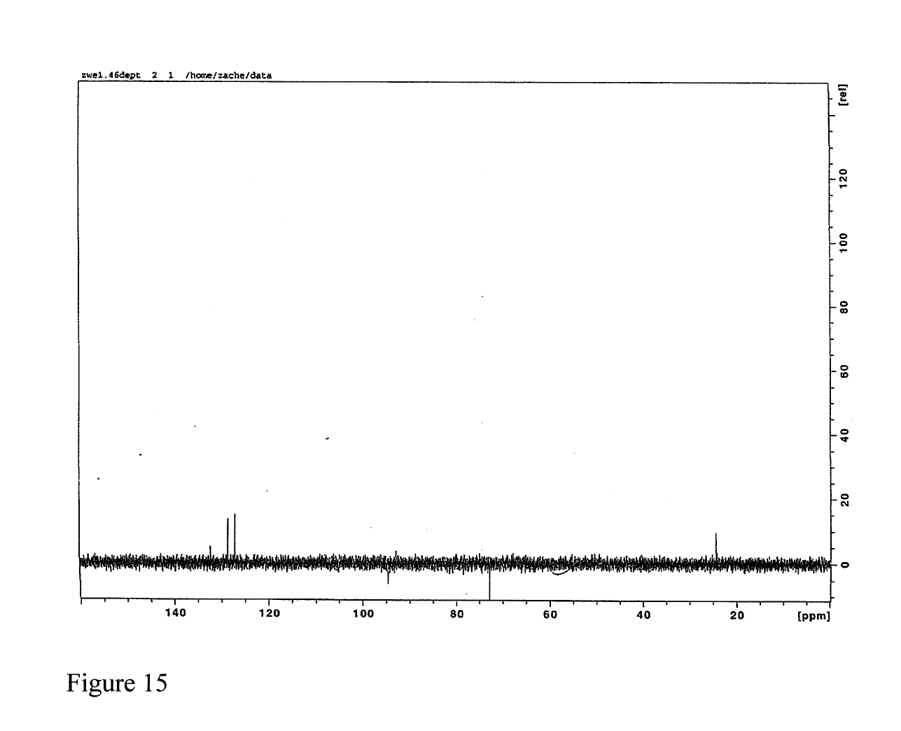

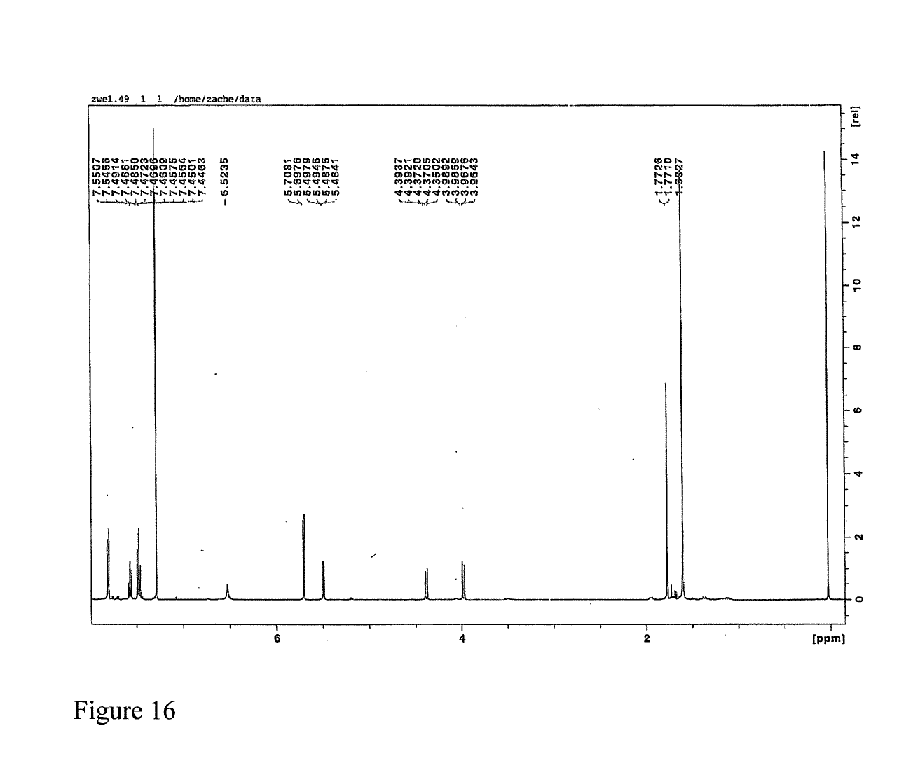

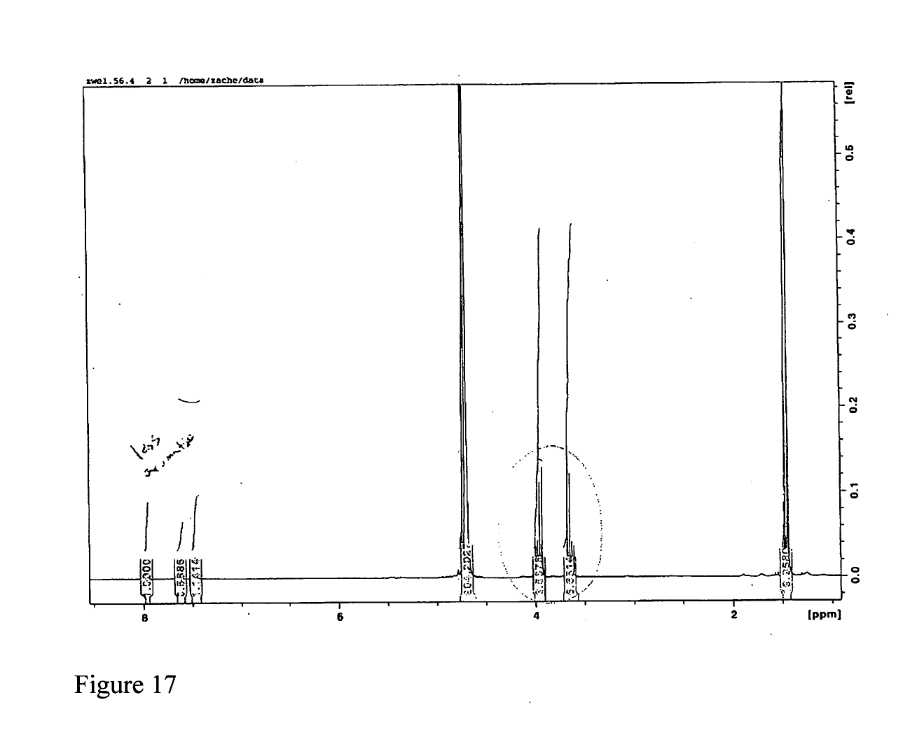

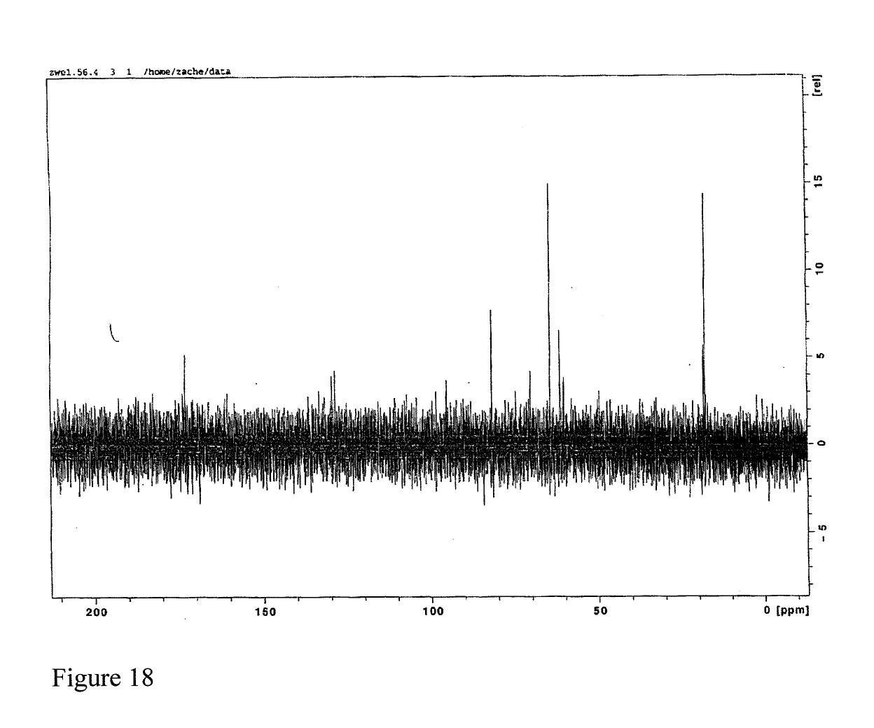

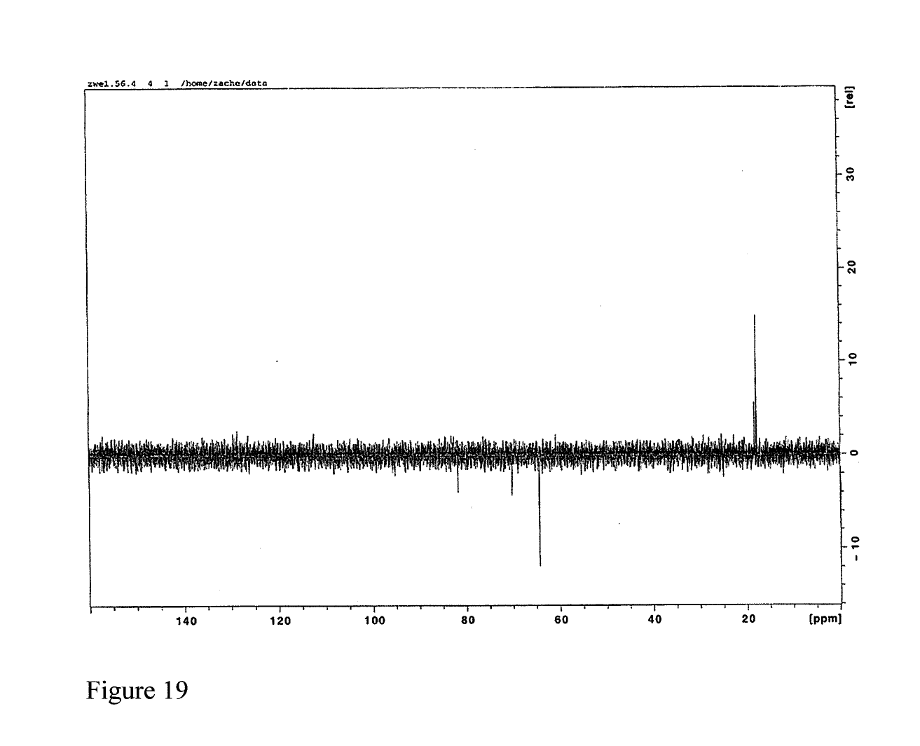

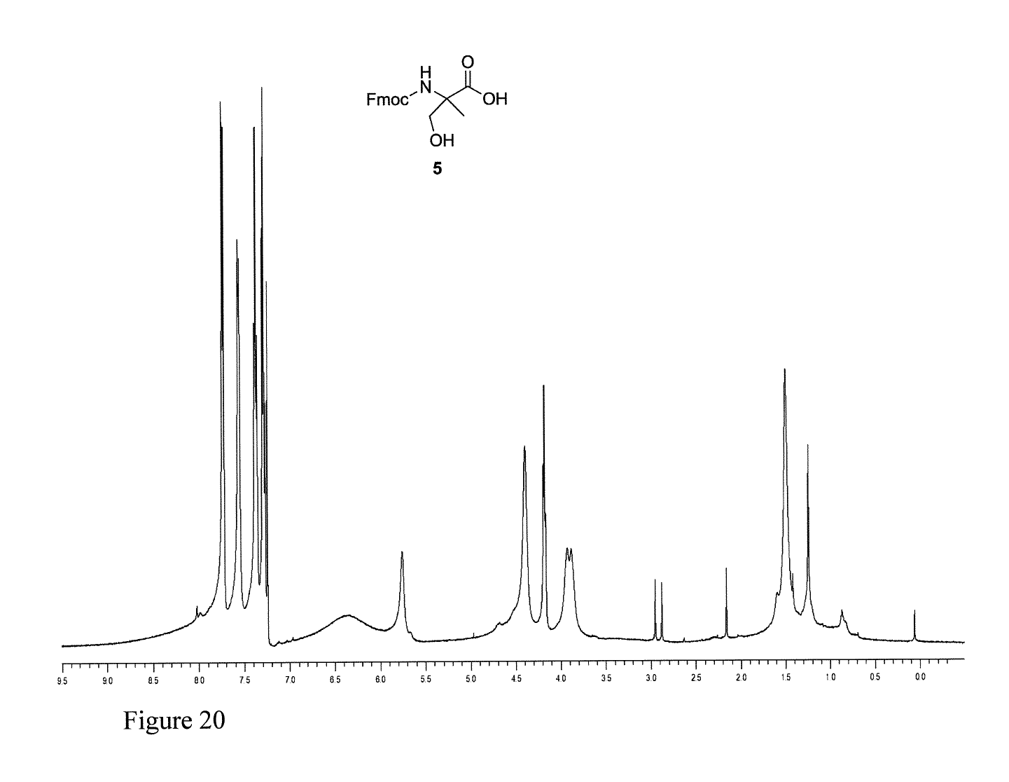

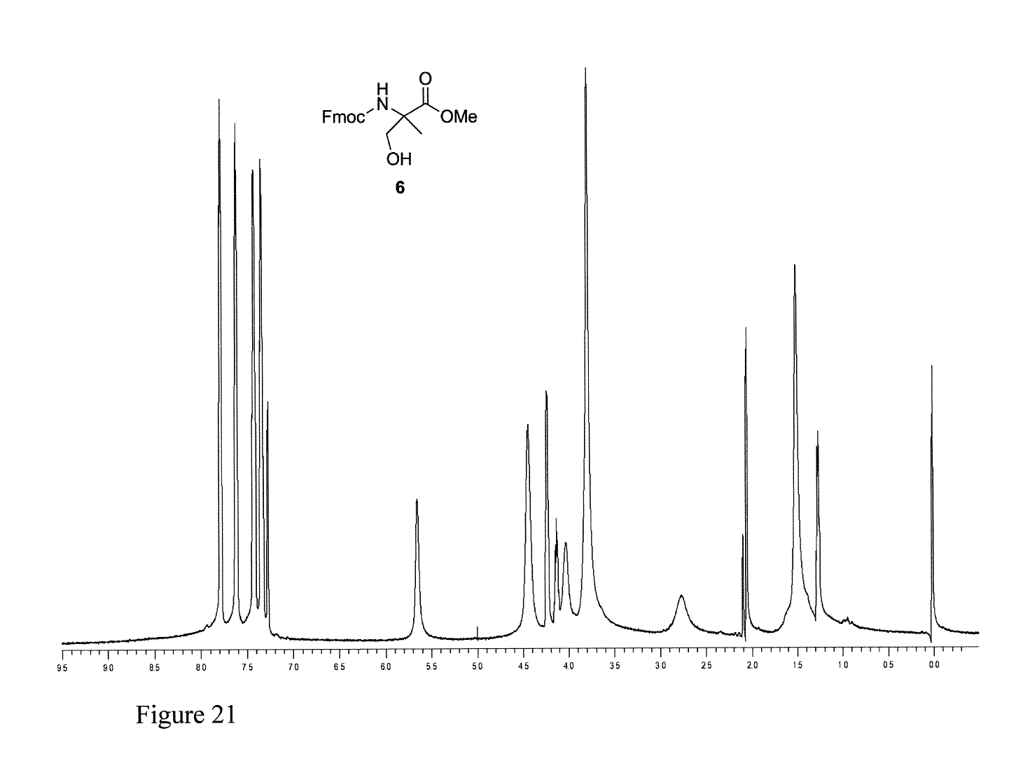

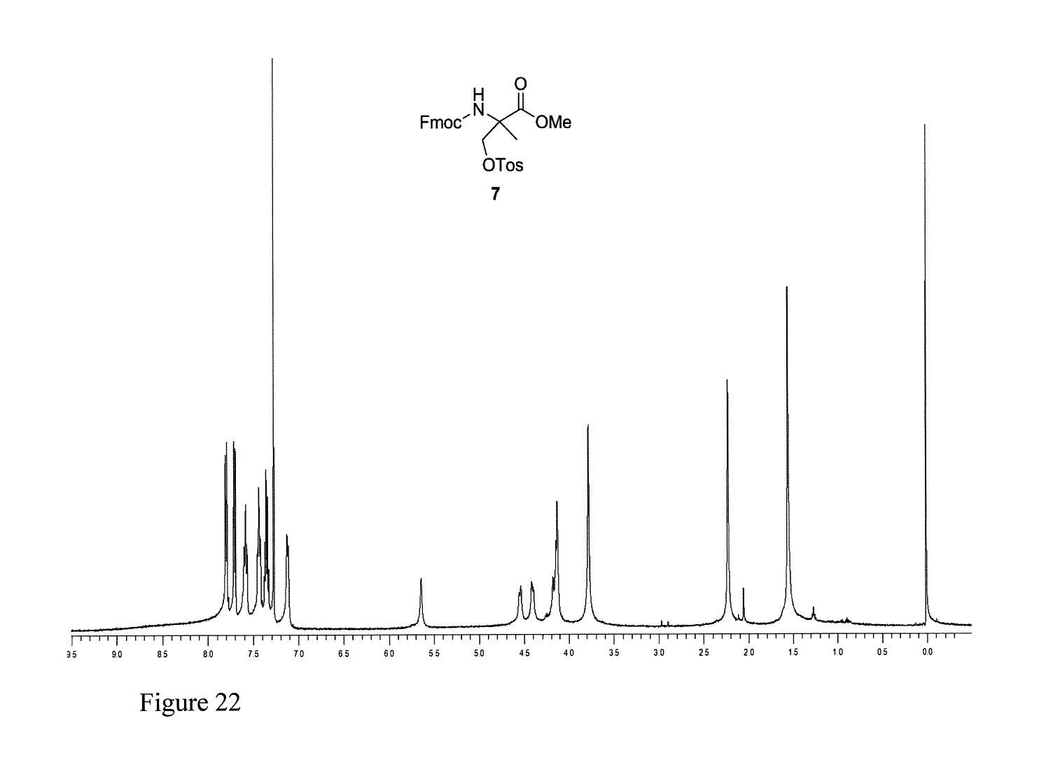

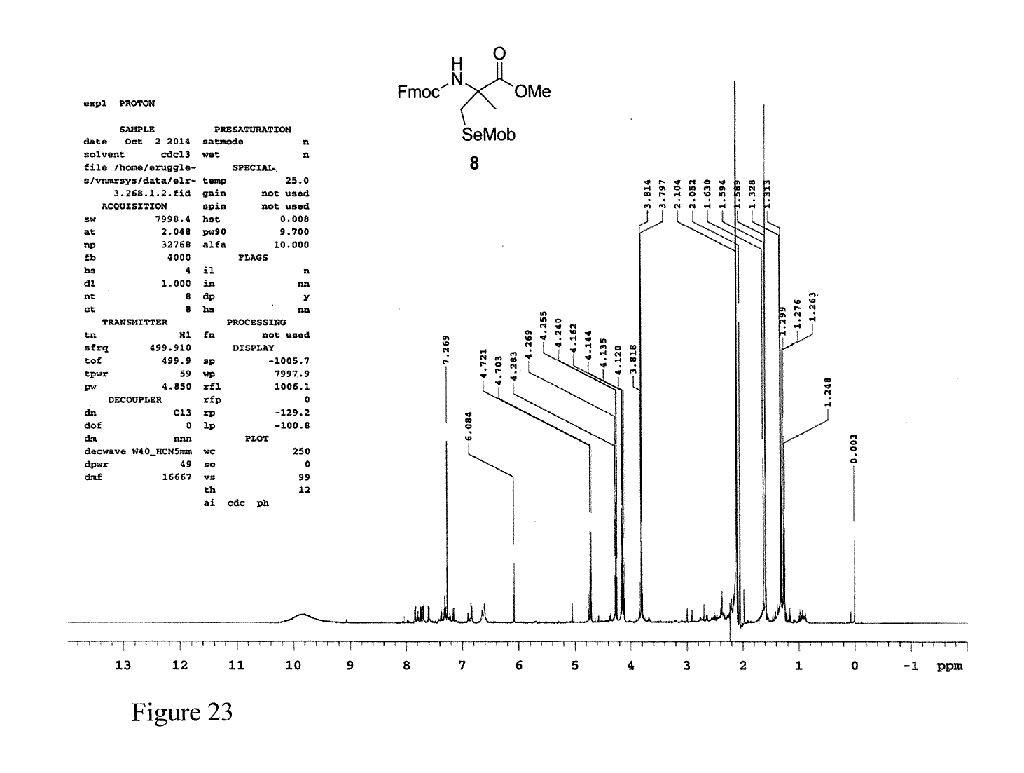

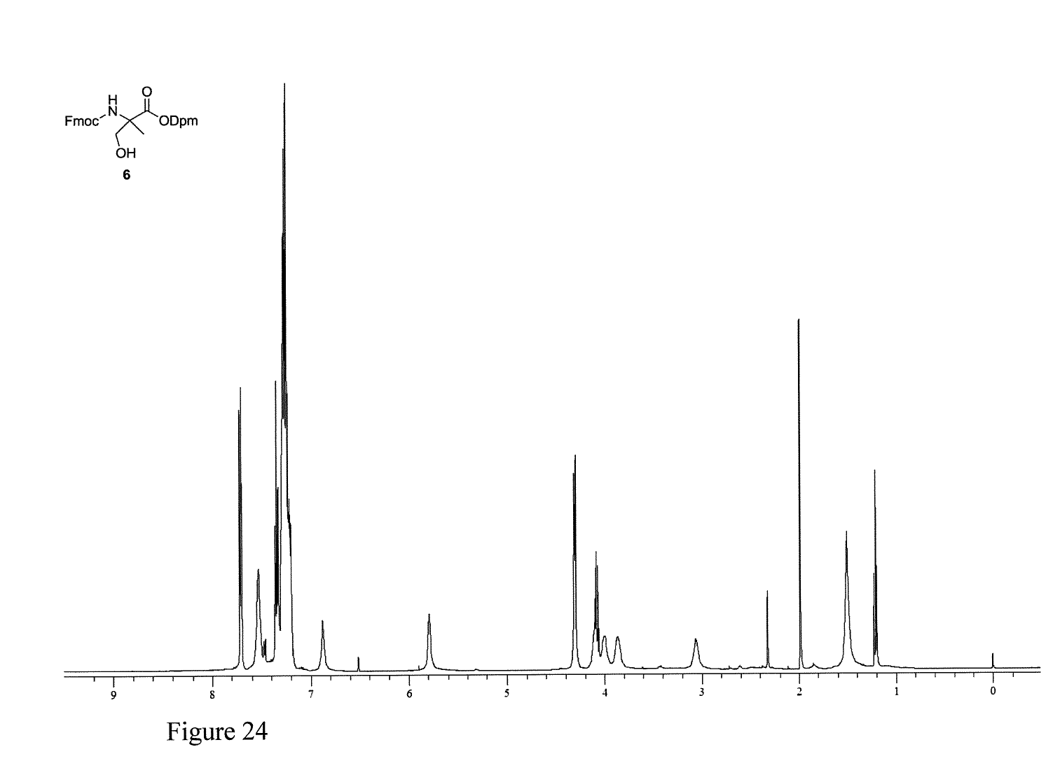

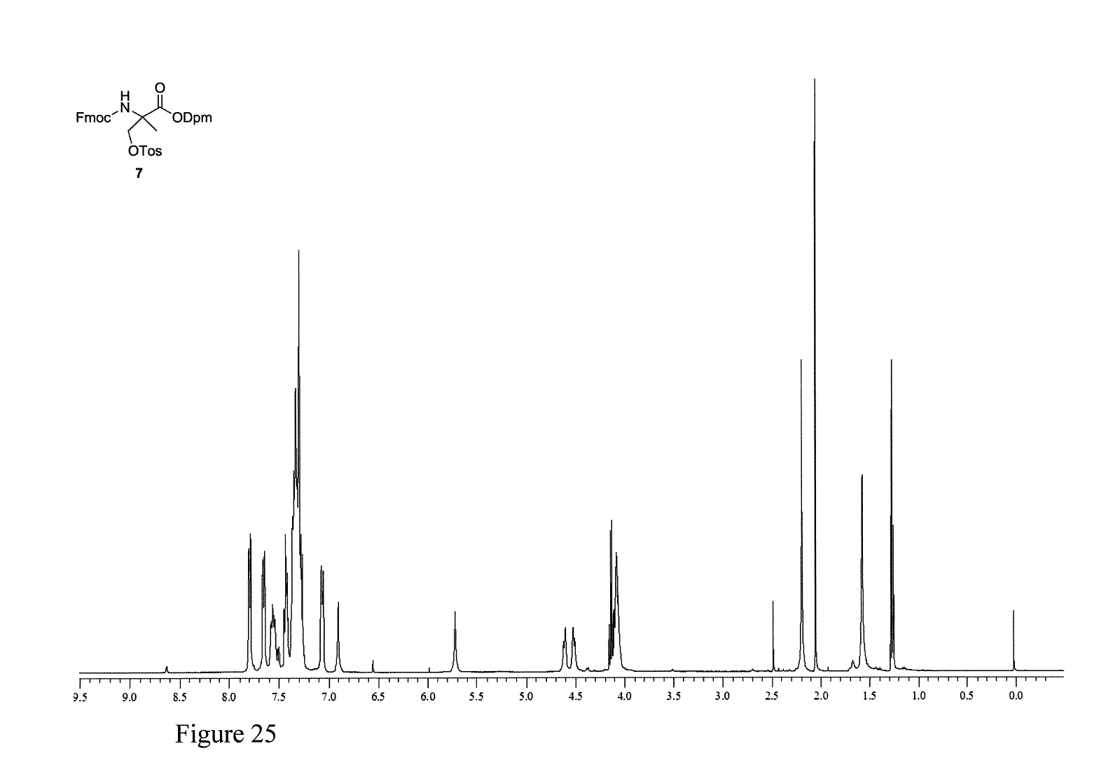

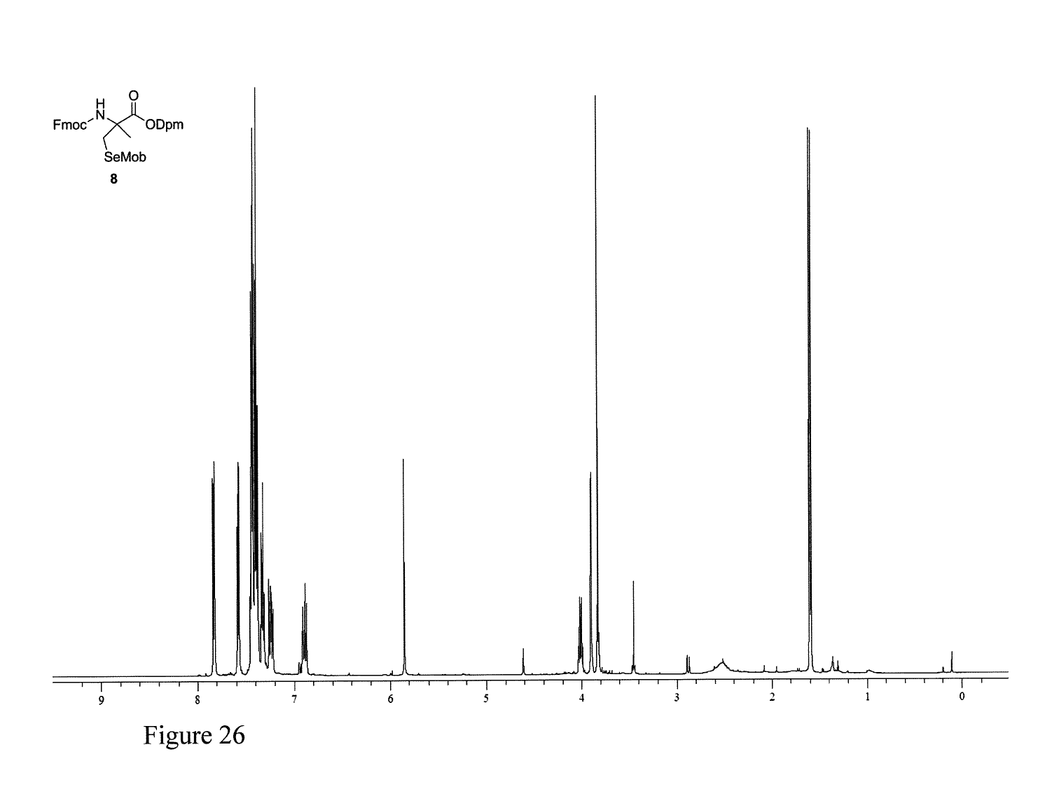

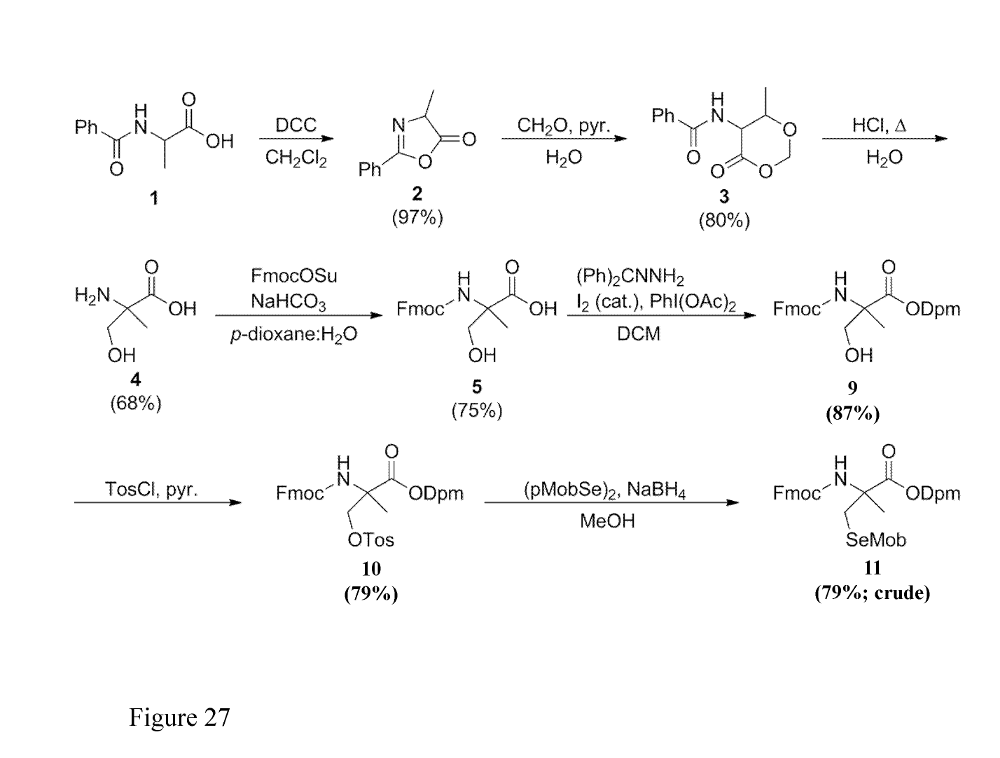

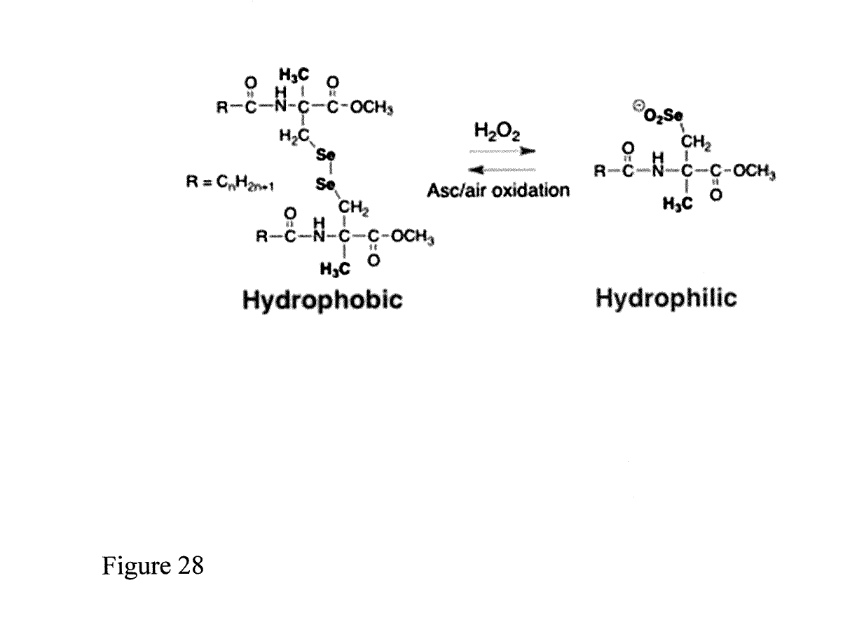

Examples of uses of uses of selenocysteine (e.g., deuterium analog or tritium analog), α-methylselenocysteine, or α-methylselenocysteine derivatives include, but are not limited to: the use of α-methyl-Se-carboxymethylselenocysteine to treat chronic inflammatory lung pathologies such as COPD, idiopathic pulmonary fibrosis, and adult respiratory distress syndrome (ARDS); the use of N-acetyl-α-methylselenocysteine to treat cystic fibrosis and autism; the use of α-methylselenocysteine as a general anti-inflammatory agent; incorporation of α-methylselenocysteine into a peptide for use as a general anti-inflammatory agent; incorporation of α-methylselenocysteine into a tripeptide analog of glutathione (FIG. 11); the use of the tripeptide analog of glutathione as a recyclable reagent for making disulfide bonds (solution phase); the use of the tripeptide analog of glutathione as a recyclable reagent for carrying out oxidation reactions (FIG. 12); the use of the tripeptide analog of glutathione as a recyclable reagent coupled to a solid-support for the use of making disulfide bonds on peptides and proteins (FIG. 12); the use of α-methylselenocysteine as part of a lipid, which will function as a redox-active switchable surfactant (FIG. 10, 28); the use of α-methylselenocysteine as an acyl transfer reagent for the purpose of making thioester bonds, which can be used in peptide ligation reactions (FIG. 8 and FIG. 9); incorporation of α-methylselenocysteine into thioredoxin reductase and other selenoenzymes so as to make these enzymes resistant to oxidative inactivation (e.g., a “super” antioxidant enzyme”); cleavable surfactant (e.g., charge silencer) for use in mass spectrometry.

In various embodiments, these compounds are used to treat cancer and diseases treated with anti-inflammatory drugs such as, for example, arthritis, fibromyalgia, cardio-vascular disease, dry-eye syndrome.

In various embodiments, these compounds are used as chemical reagents such as, for example, oxidants (e.g., regenerable oxidants) and acyl transfer reagents. In an embodiment, the compounds are attached (e.g., ionically or covalently) to a solid support to provide a solid-supported chemical reagent such an solid-supported oxidant (e.g., a solid-supported regenerable oxidant). In various embodiments, these compounds are redox-active surfactants such as, for example, redox-active switchable surfactants (e.g., for use in laundry detergent compositions). In an embodiment, the compounds are lipid soluble antioxidants. These lipid soluble antioxidants can be used as, for example, food preservatives or nutraceuticals (e.g., a source of dietary selenium). In an embodiment, a food or food composition comprises one or more of the compounds in an amount suitable to act as an antioxidant or as a dietary source of selenium.

In various embodiments, these compounds are oxidatively reversible surfactants.

The surfactant behavior of the compounds can be turned off or on by addition of a reducing agent (e.g. a charge silencer that silences the charge of the compound) or an oxidizing agent. The compound (acting as a surfactant) can may help solubilize compounds present in a mixture and the compound surfactant behavior can be “turned off” other, for analysis of the mixture by mass spectrometry (e.g., MALDI mass spectrometry).

In an embodiment, a method of making a composition comprising one or more aqueous soluble peptide fragments of a hydrophobic peptide comprises: a)forming an aqueous solution or suspension comprising: i) a compound having the following structure:

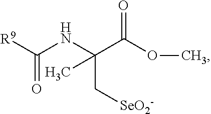

where R₉ is a C₂ to C₁₈ aliphatic group, and ii) a hydrophobic protein, b) digesting the hydrophobic protein with a protease (e.g., trypsin and chymotrypsin) to form one or more aqueous soluble peptide fragments; and c) reducing the compound (e.g., by addition of a reducing agent such as ascorbic acid, a thiol, or phosphine) to form a diselenide product and the composition comprising the one or more aqueous soluble peptide fragments of the hydrophobic protein. The method can further comprise analyzing the composition from c) by mass spectrometry. The method can further comprise: d) removing the diseleide product from the solution/suspension formed in c) (e.g., by filtration or centrifugation and decantation); e) forming a (e.g., reforming the) seleninic acid analog of the diselenide product from d) (e.g., by addition of an oxidizing agent such as hydrogen peroxide or organic peroxide (such as, for example, meta-chloroperoxybenzoic acid); f) optionally, repeating a)-d); and g) optionally, analyzing the composition from f) by mass spectrometry. The amount of compound in the aqueous solution or suspension is at least sufficient to form a aqueous solution or suspension of the hydrophobic protein. For example, the compound is present in the solution at a concentration of 1×10⁻⁶ M to 1 M. In another example, the compound is present in the solution at a concentration of 0.05 mM to 50 mM. The aqueous solution or suspension can have a pH of 4 to 8, including all 0.1 pH values and ranges therebetween. For example, the aqueous solution or suspension has a pH of 4 to 5.5. It may be desirable to use ascorbic acid as the reducing agent as it is chemoselective for selenininc acid at acidic pH.

The following examples are presented to illustrate the present disclosure. They are not intended to limiting in any manner.

### EXAMPLE 1

This example provides a description of synthetic methodology for making the compounds of the present disclosure.

Reactions employed oven-dried glassware under argon unless otherwise noted. Argon was passed through a column of anhydrous CaSO₄ before use. Chemicals were purchased from either Sigma-Aldrich (Milwaukee, Wis.) or Fisher Scientific (Pittsburgh, Pa.) and were used as received or purified by standard procedures. Reactions were monitored by thin layer chromatography (TLC) using glass 0.25 mm silica gel plates with UV indicator. Flash chromatography was performed using columns packed with 230-400 mesh silica gel as a slurry in the elution solvent, unless otherwise noted. Gradient flash chromatography was conducted by adsorption of product mixtures on silica gel, packing onto fresh silica bed as a slurry in minimal hexanes, and eluting with a continuous gradient as noted in parenthesizes. Proton and carbon NMR data were obtained with a Varian or Bruker ARX 500 spectrometer at 20° C. unless otherwise noted. Chemical shifts for ¹H NMR and ¹³C NMR are reported in parts per million (ppm) relative to tetramethylsilane (δ=0.00 ppm for ¹H NMR) or chloroform-d (δ=77.0 ppm for ¹³C NMR) respectively.

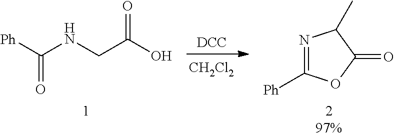

Synthesis of 4-methyl-2-phenyloxazol-5(4H)-one (2). 8.86 g (45.9 mmol, 1.00 eq.) of 1 was dissolved in CH₂Cl₂ (0.31 M) under Ar, and then chilled to 0° C. prior to the slow addition of 9.94 g (48.2 mmol, 1.05 eq.) of DCC over 15 min. After complete addition the reaction was stirred at 0° C. for 30 min, then warmed naturally to room temperature over 40 min. The reaction was vacuum-filtered and the white solid obtained was triturated with cold ether, to yield 7.65 g (96.8% yield) of 2.

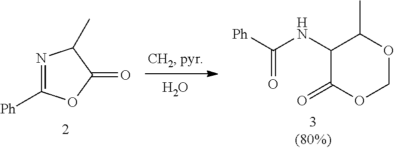

Synthesis of N-(4-methyl-6-oxo-1,3-dioxan-5-yl)benzamide (3). 7.65 g (44.5 mmol, 1.00 eq.) of 2 was dissolved in 9.85 mL (118.8 mmol, 2.67 eq.) of pyridine prior to the addition of 24.0 mL (870.8 mmol, 19.6 eq.) of formaldehyde. Reaction stirred at room temperature for 30 min. After the addition of 96.5 mL H₂O the reaction was stirred an additional 30 min. The reaction was vacuum-filtered and the resulting solid was triturated with cold H₂O to yield 8.95 g (79.5%) of 3 as a white solid.

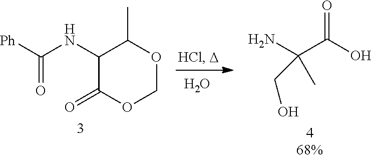

Synthesis of 2-amino-3-hydroxy-2-methylpropanoic acid (4). At room temperature, 8.95 g (35.3 mmol, 1.00 eq) of 3 was combined with 100 mL of 5.0 M HCl_{(aq)}. The reaction was brought to reflux, which was maintained for 3 hours. The reaction was cooled to room temperature prior to vacuum-filtration. The aqueous liquor was frozen and lyophilized to produce 2.85 g (67.8%) of 4 as a white solid.

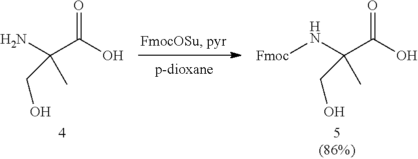

Synthesis of 2-((((9H-fluoren-9-yl)methoxy)carbonyl)amino)-3-hydroxy-2-methylpropanoic acid (5). 2.00 g (16.8 mmol, 1.00 eq.) of 4 was dissolved in 20 mL of water at room temperature and treated with 2.20 g (33.6 mmol, 2.00 eq.) NaHCO₃. Reaction was then cooled to 0° C. Separately, 8.5 g (25.2 mmol, 1.5 eq.) Fmoc-OSu was dissolved in 20 mL of 1,4-dioxane at room temperature and then chilled to 0° C. The Fmoc-OSu solution was then added to the aqueous reaction and was vigorously mixed for 1 hr. at 0° C. Allowed to warm naturally to room temperature and stirred for 12 hrs. The reaction was extracted twice with 50 mL EtOAc. The combined EtOAc extractions were washed twice with 50 mL sat. NaHCO_{3(aq)}. The aqueous layers were combined and with the addition of 12 M HCl_{(aq)} the pH was brought down to 1. The aqueous was extracted three times with 50 mL EtOAc. The EtOAc extractions were combined, dried over MgSO₄, filtered and concentrated to produce 4.93 g (86.0%) of 5 as a white solid.

Alternative synthesis of 2-((((9H-fluoren-9-yl)methoxy)carbonyl)amino)-3-hydroxy-2-methylpropanoic acid (5). 2.81 g (23.5 mmol, 1.00 eq.) of 4 was dissolved in 40 mL of water at room temperature and treated with 3.95 g (47.0 mmol, 2.00 eq.) NaHCO₃ to produce a pH=8.0. Reaction was then cooled to 0° C. Separately, 8.72 g (25.8 mmol, 1.1 eq.) Fmoc-OSu was dissolved in 100 mL of 1,4-dioxane at room temperature. The Fmoc-OSu solution was then added to the aqueous reaction via an addition funnel and was vigorously mixed for 1 hr. at 0° C. Upon warming to room temperature, an additional 50 mL of 1,4-dioxane was added and the reaction was stirred for 4 days. The reaction was partitioned with 100 mL EtOAc. The EtOAc layer was washed two times with 100 mL sat. NaHCO_{3(aq)}. The combined aqueous washes was acidified to a pH=1 with 10% HCl_{(aq)}. The aqueous layer was then extracted three times with 100 mL EtOAc. The combined EtOAc extractions were combined, dried over MgSO₄, filtered and concentrated. The white gummy solid was purified via flash silica chromatography (5% MeOH:DCM) to produce 6.02 g (75.0%) of 5 as a white solid.

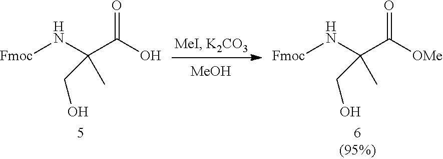

Synthesis of methyl 2-((((9H-fluoren-9-yl)methoxy)carbonyl)amino)-3-hydroxy-2-methylpropanoate (6). 369 mg (1.08 mmol, 1.00 eq.) of 5 was dissolved in DMF and then chilled to 0° C., prior to the addition of 164 mg (1.19 mmol, 1.10 eq.) K₂CO₃. Reaction stirred at 0° C. for 10 min. prior to the addition of 135 mL (2.16 mmol, 2.00 eq.) of MeI. Reaction stirred at 0° C. for 30 min. then warmed to room temperature naturally and stirred for 1 hr. The reaction was extracted three times with 25 mL EtOAc. The combined EtOAc layers were extracted five times with 30 mL H₂O. The combined EtOAc was dried over MgSO₄, filtered, and concentrated to afford 364 mg (95.0%) of 6 as an off white gum.

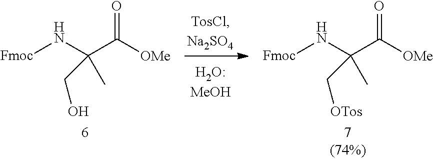

Synthesis of methyl 2-((((9H-fluoren-9-yl)methoxy)carbonyl)amino)-2-methyl-3-(tosyloxy)propanoate (7). 180 mg (0.51 mmol, 1.00 eq.) of 6 was dissolved in 6.0 mL of CH₂Cl₂ prior to the addition of 193 mg (1.01 mmol, 2.00 eq.) of TosCl and 98 microL of pyridine. The reaction was stirred at room temperature for 16 hrs. Reaction was concentrated and then dissolved in 20 mL EtOAc. The EtOAc solution was extracted three times with 1.0 M HCl_{(aq)}. The EtOAc layer was dried over MgSO₄, filtered, and concentrated. The resulting oil was purified via flash silica chromatography (4:1 Hex/EtOAc) to yield 192 mg (73.9%) of 7 as a white solid.

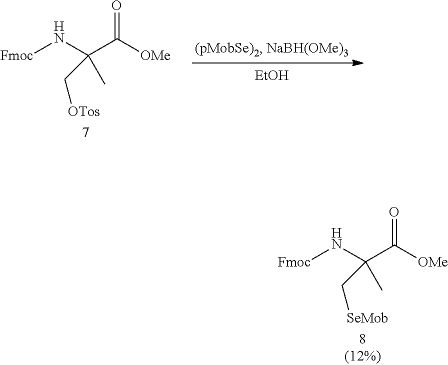

Synthesis of methyl 2-((((9H-fluoren-9-yl)methoxy)carbonyl)amino)-3-((4-methoxybenzyl)selanyl)-2-methylpropanoate (8). 104 mg (0.260 mmol, 1.00 eq.) of p-methoxybenzyldiselenide was vigorously mixed in 3.0 mL anhydrous EtOH, under Ar, for 15 min at room temperature prior to the rapid addition of 66.5 mg (0.520 mmol, 2.00 eq.) NaBH(OMe)₃. The reaction was stirred for 30 min. prior to the addition of 188 mg (0.369 mmol, 1.42 eq.) of 7. Reaction stirred for 3 hrs at room temperature and then concentrated. The residual was taken up in 25 mL ether and washed three times with sat. NH₄Cl_{(aq)}. The combined aqueous washes were extracted three times with 25 mL ether. The combined ethereal extractions were dried over MgSO₄, filtered, and concentrated to produce 16.9 mg (12.1%) of 8 as an off yellow solid.

Alternative synthetic route using a Dpm protection.

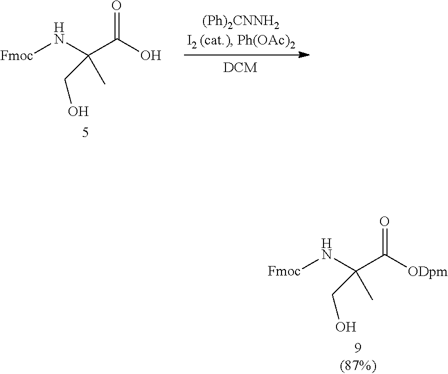

Synthesis of benzhydryl 2-((((9H-fluoren-9-yl)methoxy)carbonyl)amino)-3-hydroxy-2-methylpropanoate (9). 4.06 g (11.8 mmol, 1.00 eq.) of 5 and 3.50 g (17.8 mmol, 1.50 eq.) of (Ph)₂CNNH₂ were dissolved in 30 mL DCM under Ar at room temperature, prior to the addition of 2.0 mL of a 1% I₂/DCM (wt/v). The reaction was then chilled to −10° C. 5.74 g (17.8 mmol, 1.50 eq.) of PhI(OAc)₂ was then added over 1.5 hr in six portions. Reaction was warmed to 0° C. and stirred for 45 min. The reaction was washed with 50 mL H₂O, three times with 50 mL sat. NaHCO_{3(aq)}, and once more with 50 mL H₂O. The DCM layer was dried over MgSO₄, filtered, and concentrated to afford and orange oil. The oil was purified via flash silica chromatography (5:1 Hex/EtOAc to 100% EtOAc) to produce 5.20 g (87.0%) of 9 as an off yellow solid.

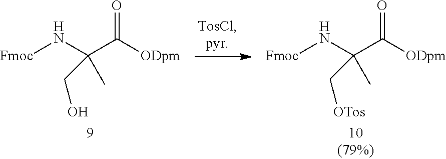

Synthesis of benzhydryl 2-((((9H-fluoren-9-yl)methoxy)carbonyl)amino)-2-methyl-3-(tosyloxy)propanoate (10). 3.08 g (6.26 mmol, 1.00 eq.) of 9 was dissolved in 23.2 mL of pyridine and cooled to 0° C. prior to the addition of 2.93 g (15.3 mmol, 2.45 eq.) of TosCl. The reaction was stirred at 0° C. for 2 hrs. The reaction was warmed to room temperature and stirred for 48 hrs. The reaction was portioned with the addition of 50 mL EtOAc and 50 mL phosphate buffer (pH=4.2). The EtOAc layer was washed five times with the phosphate buffer (pH=4.2). Reaction was concentrated and then dissolved in 20 mL EtOAc. The EtOAc layer was dried over MgSO₄, filtered, and concentrated. The resulting oil was purified via flash silica chromatography (5:1 Hex/EtOAc to 100% EtOAc) to yield 3.27 g (79.0%) of 10 as a white solid.

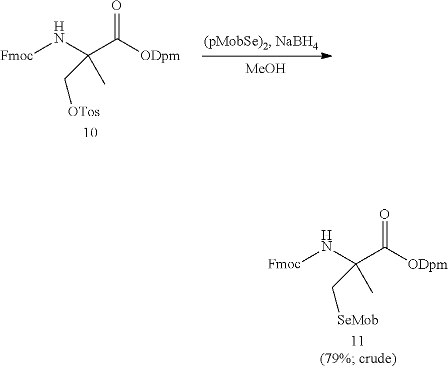

Synthesis of benzhydryl 2-((((9H-fluoren-9-yl)methoxy)carbonyl)amino)-3-((4-methoxybenzyl)selanyl)-2-methylpropanoate (11). 100.2 mg (0.250 mmol, 1.00 eq.) of p-methoxybenzyldiselenide was vigorously mixed in 5.0 mL anhydrous EtOH, under Ar, The reaction was cooled to 0° C. and 18.9 mg (0.500 mmol, 2.00 eq.) of NaBH₄. After 10 min 10 in 2 mL anhydrous EtOH and 4 mL anhydrous DMF was added dropwise to the cold reaction. After complete addition the reaction was allowed to warm naturally to room temperature. After 1 hr, the reaction was treated with an additional 30 mg NaBH₄ to ensure complete diselenide cleavage. The reaction was portioned with 50 mL water and 20 mL EtOAc. The EtOAc layer was washed once with 50 mL H₂O and 50 mL sat. NH₄Cl_{(aq)}. The EtOAc extraction was dried over MgSO₄, filtered, and concentrated to produce 0.275 g (92.0%) of 11 as a crude off yellow solid.

1. A method of making α-methylselenocysteine, a protected derivative thereof, or a derivative thereof comprising:
a) protecting the amino group, the carboxylate group, and the alcohol group of α-methylserine to form an α-methylserine having a protected amino group and protected carboxylate group;
b) converting the alcohol group of the α-methylserine to a leaving group to form an α-methylserine having a protected amino group, protected carboxylate group, and leaving group;
c) reacting the α-methylserine having a protected amino group, protected carboxylate group, and leaving group with a selenating reagent to form a protected α-methylselenocysteine having a protected amino group, protected carboxylate group, and a protected selenol group; and
d) optionally, deprotecting the protected amino group and/or the protected carboxyl group and/or protected selenol group to form α-methylselenocysteine or a protected derivative thereof.

2. The method of claim 1, wherein the selenating reagent is formed in situ during the reaction of the α-methylserine having a protected amino group, protected carboxylate group, and protected alcohol group and the selenating reagent.

3. The method of claim 1, further comprising:
e) forming a diselenide linkage between two of the protected α-methylselenocysteine molecules of c) to form a protected diselenide product; and
f) optionally, deprotecting the protected amino group and/or the protected carboxyl group of the protected diselenide product from e) to form a deprotected diselenide product;
g) reducing the protected diselenide product or deprotected diselenide product from e) or f) with a suitable reagent such that carbo-α-methylselenocysteine or a protected derivative thereof is formed.

4. The method of claim 1, further comprising:
h) forming a —H group or C₁ to C₁₈ ester group by reaction of the protected carboxylate group or deprotected carboxylate group of the α-methylselenocysteine having the protected amino group or deprotected amino group, the protected selenol group or deprotected selenol group, or a combination thereof.

5. The method of claim 1, further comprising:
i) forming one or two —H groups, one or two C₁ to C₁₈ aliphatic groups, one or two 13 C(O)R⁶ groups, wherein R⁶ is a C₁ to C₁₈ aliphatic group, or a combination thereof, by reaction of the protected amino group or deprotected amino group of the α-methylselenocysteine having a protected carboxylate group or deprotected carboxylate group, protected selenol group or deprotected selenol group.

6. The method of claim 1, further comprising:
j) forming a —H group, —CH₂COOR⁷ group, wherein R⁷ is H or CH₂R⁸, wherein R⁸ is an alkyl, aliphatic, or aromatic group, or a combination thereof, by reaction of the protected selenol group or deprotected selenol group of the α-methylselenocysteine having the protected amino group or deprotected amino group, the protected carboxylate group or deprotected carboxylate group.

7. A method of making deuterated or tritiated selenocysteine comprising:
a) transforming α-deuterated serine to α-deutero-β-chloroalanine or α-tritiated serine to α-tritio-β-chloroalanine; and
b) transforming α-deutero-β-chloroalanine to α-deutero-selenocystine or α-tritio-β-chloroalanine to α-tritio-selenocystine.

8. A method of making a composition comprising one or more aqueous soluble peptide fragments of a hydrophobic peptide comprising:
a) forming an aqueous solution or suspension comprising:
i) a compound having the following structure: 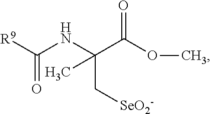
wherein R₉ is a C₂ to C₁₈ aliphatic group, and
ii) a hydrophobic protein,
b) digesting the hydrophobic protein with a protease to form one or more aqueous soluble peptide fragments; and
c) reducing the compound to form a diselenide product and the composition comprising the one or more aqueous soluble peptide fragments of the hydrophobic protein.

9. The method of claim 8, further comprising analyzing the composition from c) by mass spectrometry.

10. The method of claim 8, further comprising:
d) removing the diseleide product from the solution/suspension formed in c);
e) forming a seleninic acid analog of the diselenide product from d);
f) optionally, repeating a)-d); and
g) optionally, analyzing the composition from f) by mass spectrometry.

11. A method of making a disulfide bond comprising contacting a protein and/or peptide-that is the same or different that is capable of forming a disulfide bond with a reagent comprising a compound having the following structure: 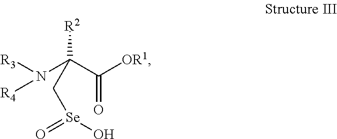
wherein R¹ is selected from hydrogen atom (H), C₁ to C₁₈ alkyl groups, and protecting group; R² is selected from deuterium atom (D), tritium (T), and a methyl group; R³ and R⁴ are each independently selected from hydrogen atom (H), C₁ to C₁₈ aliphatic groups, protecting group, and —C(O)R⁶, where R⁶ is a C₁ to C₁₈ aliphatic group; R⁵ is hydrogen atom (H), protecting group, or CH₂COOR⁷, where R⁷ is H or CH₂R⁸, where R⁸ is an alkyl, aliphatic, or aromatic group, or
a moiety derived from the compound, or
a composition comprising the reagent,
to form a disulfide bond.

12. The method of claim 11, wherein the reagent is coupled to a solid support.

13. A method of making a thioester bond in a protein or peptide comprising contacting a protein and/or peptide that is the same or different that is capable of forming a thioester bond with a reagent comprising a compound having the following structure: 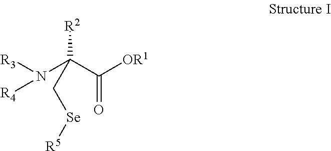
or
wherein R¹ is selected from hydrogen atom (H), C₁ to C₁₈ alkyl groups, and protecting group; R² is selected from deuterium atom (D), tritium (T), and a methyl group; R³ and R⁴ are each hydrogen atom (H); R⁵ is hydrogen atom (H), or a moiety derived from the compound, or
a composition comprising the reagent,
to form a thioester bond.

14. The method of claim 13, wherein the reagent is coupled to a solid support.
